(19) United States
(12) Patent Application Publication
(10) Pub. No.: US 2018/0085341 A1
(43) Pub. Date: Mar. 29, 2018

(54) METHODS FOR TREATING CANCER

(71) Applicant: BOSTON BIOMEDICAL, INC., Cambridge, MA (US)

(72) Inventors: Chiang J. LI, Cambridge, MA (US); Wei LI, Wayland, MA (US); Youzhi LI, Westwood, MA (US); Matthew J. Hitron, West Roxbury, MA (US); Yuan GAO, Belmont, MA (US)

(73) Assignee: BOSTON BIOMEDICAL, INC., Cambridge, MA (US)

(21) Appl. No.: 15/566,919

(22) PCT Filed: Apr. 18, 2016
(86) PCT No.: PCT/US2016/028177

§ 371 Date: Oct. 16, 2017

Related U.S. Application Data
(60) Provisional application No. 62/149,349, filed on Apr. 17, 2015.
(60) Provisional application No. 62/280,947, filed on Jan. 20, 2016.

(51) Int. Cl. A61K 31/343; A61K 31/337

(52) U.S. Cl. CPC A61K 31/343; A61K 2300/00; A61K 31/337

(57) ABSTRACT

Methods comprising administering and kits comprising at least one paclitaxel compound and at least one compound of formula (I).

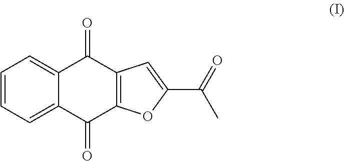 [SMILES: CC(=O)c1cc2c(o1)C(=O)c1ccccc1C2=O]

The present application claims the benefit of priority under 35 U.S.C. § 119 of U.S. Provisional Patent Application No. 62/149,349, filed Apr. 17, 2015, and U.S. Provisional Patent Application No. 62/280,947 filed Jan. 20, 2016; the content of each respective application is incorporated herein by reference.

Disclosed herein are methods comprising administering to a subject a combination comprising a therapeutically effective amount of at least one compound chosen of formula (I) in combination with a therapeutically effective amount of at least one paclitaxel compound chosen from paclitaxel, pharmaceutically acceptable salts thereof, and solvates of any of the foregoing.

The at least one compound of formula (I) is chosen from compounds having formula (I)

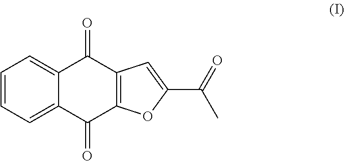 [SMILES: CC(=O)c1cc2c(o1)C(=O)c1ccccc1C2=O]

prodrugs, derivatives, pharmaceutically acceptable salts of any of the foregoing, and solvates of any of the foregoing.

Cancer fatalities in the United States alone number in the hundreds of thousands each year. Despite advances in the treatment of certain forms of cancer through surgery, radiotherapy, and chemotherapy, many types of cancer are essentially incurable. Even when an effective treatment is available for a particular cancer, the side effects of such treatment can be severe and result in a significant decrease in quality of life.

Most conventional chemotherapy agents have toxicity and limited efficacy, particularly for patients with advanced solid tumors. Conventional chemotherapeutic agents cause damage to non-cancerous as well as cancerous cells. The therapeutic index (i.e., a measure of a therapy's ability to discriminate between cancerous and normal cells) of such chemotherapeutic compounds can be quite low. Frequently, a dose of a chemotherapy drug that is effective to kill cancer cells will also kill normal cells, especially those normal cells (such as epithelial cells and cells of the bone marrow) that undergo frequent cell division. When normal cells are affected by the therapy, side effects such as hair loss, suppression of hematopoiesis, and nausea can occur. Depending on the general health of a patient, such side effects can preclude the administration of chemotherapy, or, at least, be extremely unpleasant and uncomfortable for the patient and severely decrease quality of the remaining life of cancer patients. Even for cancer patients who respond to chemotherapy with tumor regression, cancers often quickly relapse, progress and form more metastasis after initial response to chemotherapy. Such recurrent cancers become highly resistant or refractory to chemotherapeutics. As discussed below, cancer stem cells (CSCs) or cancer cells with high stemness (stemness-high cancer cells) are responsible for the rapid tumor recurrence and resistance to further traditional chemotherapy.

CSCs are believed to possess the following four characteristics:

1. Stemness—As used herein, stemness means the capacity to self-renew and differentiate into cancer cells (Gupta P B et al., Nat. Med. 2009; 15(9):1010-1012). While CSCs are only a minor portion of the total cancer cell population (Clarke M F, Biol. Blood Marrow Transplant. 2009; 11(2 suppl 2):14-16), they can give rise to heterogeneous lineages of cancer cells that make up the bulk of the tumor (see Gupta et al. 2009). In addition, CSCs possess the ability to mobilize to distinct sites while retaining their stemness properties and thus regrowth of the tumor at these sites (Jordan C T et al. N. Engl. J. Med. 2006; 355(12):1253-1261).

2. Aberrant signaling pathways—CSC stemness is associated with dysregulation of signaling pathways, which may contribute to their ability to regrow tumors and to migrate to distant sites. In normal stem cells, stemness signaling pathways are tightly controlled and genetically intact. In contrast, stemness signaling pathways in CSCs are dysregulated, allowing these cells to self-renew and differentiate into cancer cells (see Ajani et al. 2015). Dysregulation of stemness signaling pathways contributes to CSC resistance to chemotherapy and radiotherapy and to cancer recurrence and metastasis. Exemplary stemness signaling pathways involved in the induction and maintenance of stemness in CSCs include: JAK/STAT, Wnt/β-catenin, Hedgehog, Notch, and Nanog (Boman B M et al., J. Clin. Oncol. 2008; 26(17):2828-2838).

3. Resistance to traditional therapies—evidence suggests that CSCs possess resistance to conventional chemotherapy and radiation. While the detailed mechanism underlying such resistance is not well understood, the stemness pathways of CSCs (see Boman et al. 2008) together with the tumor microenvironment and aberrant regulation of signaling pathways (Borovski T. et al., Cancer Res. 2011; 71(3):634-639) may contribute to such resistance.

4. Ability to contribute to tumor recurrence and metastasis—although chemotherapy and radiation may kill most of the cells in a tumor, since CSCs are resistant to traditional therapies, the CSCs that are not eradicated may lead to regrowth or recurrence of the tumor either at the primary site or at distant sites (see Jordan et al. 2006). As mentioned above, CSCs may acquire the ability to mobilize to different sites and may maintain stemness at these sites through interactions with the microenvironment, allowing for metastatic tumor growth (see Boman et al. 2008).

The transcription factor Signal Transducer and Activator of Transcription 3 (referred to herein as Stat3) is a member of the Stat family, which are latent transcription factors activated in response to cytokines/growth factors to promote proliferation, survival, and other biological processes. Stat3 is an oncogene that can be activated by phosphorylation of a critical tyrosine residue mediated by growth factor receptor tyrosine kinases, including but not limited to, e.g., Janus kinases (JAKs), Src family kinases, EGFR, Abl, KDR, c-Met, and Her2. Yu, H. Stat3: Linking oncogenesis with tumor immune evasion in AACR 2008 Annual Meeting. 2008. San Diego, Calif. Upon tyrosine phosphorylation, the phosphorylated Stat3 (“pStat3”) forms homo-dimers and translocates to the nucleus, where it binds to specific DNA-response elements in the promoters of target genes, and induces gene expression. Pedranzini, L., et al. J. Clin. Invest., 2004. 114(5): p. 619-22.

In normal cells, Stat3 activation is transient and tightly regulated, lasting for example from 30 minutes to several hours. However, Stat3 is found to be aberrantly active in a wide variety of human cancers, including all the major carcinomas as well as some hematologic tumors. Persistently active Stat3 occurs in more than half of breast and lung cancers, colorectal cancers (CRC), ovarian cancers, hepatocellular carcinomas, multiple myelomas, etc., and in more than 95% of head/neck cancers. Stat3 plays multiple roles in cancer progression and is considered to be one of the major mechanisms for drug resistance to cancer cells. As a potent transcription regulator, Stat3 targets genes involved in cell cycle, cell survival, oncogenesis, tumor invasion, and metastasis, such as Bcl-xl, c-Myc, cyclin D1, Vegf, MMP-2, and survivin. Catlett-Falcone, R., et al. Immunity, 1999. 10(1): p. 105-15; Bromberg, J. F., et al. Cell, 1999. 98(3): p. 295-303; Kanda, N., et al. Oncogene, 2004. 23(28): p. 4921-29; Schlette, E. J., et al. J Clin Oncol, 2004. 22(9): p. 1682-88; Niu, G., et al. Oncogene, 2002. 21(13): p. 2000-08; Xie, T. X., et al. Oncogene, 2004. 23(20): p. 3550-60. It is also a key negative regulator of tumor immune surveillance and immune cell recruitment. Kortylewski, M., et al. Nat. Med., 2005. 11(12): p. 1314-21; Burdelya, L., et al. J. Immunol., 2005. 174(7): p. 3925-31; and Wang, T., et al. Nat. Med., 2004. 10(1): p. 48-54.

Abrogation of Stat3 signaling by using anti-sense oligonucleotides, siRNA, dominant-negative form of Stat3, and/or the targeted inhibition of tyrosine kinase activity causes cancer cell-growth arrest, apoptosis, and reduction of metastasis frequency both in vitro and/or in vivo. Pedranzini, L., et al. J Clin. Invest., 2004. 114(5): p. 619-22; Bromberg, J. F., et al. Cell, 1999. 98(3): p. 295-303; Darnell, J. E. Nat. Med., 2005. 11(6): p. 595-96; and Zhang, L., et al. Cancer Res, 2007. 67(12): p. 5859-64.

Furthermore, Stat 3 may play a role in the survival and self-renewal capacity of CSCs across a broad spectrum of cancers. Therefore, an agent with activity against CSCs may hold great promise for cancer patients (Boman, B. M., et al. J. Clin. Oncol. 2008. 26(17): p. 2795-99).

As discussed above, CSCs are a sub-population of cancer cells (found within solid tumors or hematological cancers) that possess characteristics normally associated with stem cells. These cells can grow faster after reduction of non-stem regular cancer cells by chemotherapy, which may be the mechanism for quick relapse after chemotherapies. In contrast to the bulk of cancer cells, which are non-tumorigenic, CSCs are tumorigenic (tumor-forming). In human acute myeloid leukemia, the frequency of these cells is less than 1 in 10,000. Bonnet, D. and J. E. Dick. Nat. Med., 1997. 3(7): p. 730-37. There is mounting evidence that such cells exist in almost all tumor types. However, as cancer cell lines are selected from a sub-population of cancer cells that are specifically adapted to growth in tissue culture, the biological and functional properties of these cell lines can change dramatically. Therefore, not all cancer cell lines contain CSCs.

CSCs have stem cell properties such as self-renewal and the ability to differentiate into multiple cell types. They persist in tumors as a distinct population and they give rise to the differentiated cells that form the bulk of the tumor mass and phenotypically characterize the disease. CSCs have been demonstrated to be fundamentally responsible for carcinogenesis, cancer metastasis, cancer recurrence, and relapse. CSCs are also called, for example, tumor initiating cells, cancer stem-like cells, stem-like cancer cells, highly tumorigenic cells, or super malignant cells.

CSCs are inherently resistant to conventional chemotherapies, which means they are left behind by conventional therapies that kill the bulk of tumor cells. As such, the existence of CSCs has several implications in terms of cancer treatment and therapy. These include, for example, disease identification, selective drug targets, prevention of cancer metastasis and recurrence, treatment of cancer refractory to chemotherapy and/or radiotherapy, treatment of cancers inherently resistant to chemotherapy or radiotherapy and development of new strategies in fighting cancer.

The efficacy of cancer treatments are, in the initial stages of testing, often measured by the amount of tumor mass they kill off. As CSCs form a very small proportion of the tumor cell population and have markedly different biologic characteristics than their differentiated progeny, the measurement of tumor mass may not select for drugs that act specifically on the stem cells. In fact, CSCs are radio-resistant and refractory to chemotherapeutic and targeted drugs. Normal somatic stem cells are naturally resistant to chemotherapeutic agents—they have various pumps (e.g., multidrug resistance protein pump) that efflux drugs, higher DNA repair capability, and have a slow rate of cell turnover (chemotherapeutic agents naturally target rapidly replicating cells). CSCs, being the mutated counterparts of normal stem cells, may also have similar functions that allow them to survive therapy. In other words, conventional chemotherapies kill differentiated (or differentiating) cells, which form the bulk of the tumor that is unable to generate new cells. A population of CSCs that gave rise to the tumor could remain untouched and cause a relapse of the disease. Furthermore, treatment with chemotherapeutic agents may only leave chemotherapy-resistant CSCs, so that the ensuing tumor will most likely also be resistant to chemotherapy. Cancer stem cells have also been demonstrated to be resistant to radiation therapy (XRT). Hambardzumyan, et al. Cancer Cell, 2006. 10(6): p. 454-56; and Baumann, M., et al. Nat. Rev. Cancer, 2008. 8(7): p. 545-54.

Since surviving CSCs can repopulate the tumor and cause relapse, anti-cancer therapies that include strategies against CSCs hold great promise. Jones R J et al., J Natl Cancer Inst. 2004; 96(8):583-585. By targeting CSC pathways, it may be possible to treat patients with aggressive, non-resectable tumors and refractory or recurrent cancers as well as prevent tumor metastasis and recurrence. Development of specific therapies targeting CSCpathways, therefore, may improve the survival and quality of life of cancer patients, especially those patients suffering from metastatic disease. Unlocking this untapped potential may involve the identification and validation of pathways that are selectively important for CSC self-renewal and survival. Though multiple pathways underlying tumorigenesis in cancer and in embryonic stem cells or adult stem cells have been elucidated in the past, pathways for cancer stem cell self-renewal and survival are still sought.

Methods for identification and isolation of CSCs have been reported. The methods used mainly exploit the ability of CSCs to efflux drugs or have been based on the expression of surface markers associated with cancer stem cells.

For example, since CSCs are resistant to many chemotherapeutic agents, it is not surprising that CSCs almost ubiquitously overexpress drug efflux pumps such as ABCG2 (BCRP-1), and other ATP binding cassette (ABC) superfamily members. Ho, M. M., et al. Cancer Res., 2007. 67(10): p. 4827-33; Wang, J., et al. Cancer Res., 2007. 67(8): p. 3716-24; Haraguchi, N., et al. Stem Cells, 2006. 24(3): p. 506-13; Doyle, L. A. and D. D. Ross. Oncogene, 2003. 22(47): p. 7340-58; Alvi, A. J., et al. Breast Cancer Res., 2003. 5(1): p. R1-R8; Frank, N. Y., et al. Cancer Res., 2005. 65(10): p. 4320-33; and Schatton, T., et al. Nature, 2008. 451(7176): p. 345-49. Accordingly, the side population (SP) technique, originally used to enrich hematopoetic and leukemic stem cells, was also employed to identify and isolate CSCs. Kondo, T., et al. Proc. Natl Acad. Sci. USA, 2004. 101(3): p. 781-86. This technique, first described by Goodell et al., takes advantage of differential ABC transporter-dependent efflux of fluorescent dyes such as Hoechst 33342 to define a cell population enriched in CSCs. Doyle, L. A. and D. D. Ross. Oncogene, 2003. 22(47): p. 7340-58; and Goodell, M. A., et al. J. Exp. Med., 1996. 183(4): p. 1797-806. Specifically, the SP is revealed by blocking drug efflux with verapamil,at which point the dyes can no longer be pumped out of the SP.

Efforts have also focused on finding specific markers that distinguish CSCs from the bulk of the tumor. Markers originally associated with normal adult stem cells have been found to also mark CSCs and co-segregate with the enhanced tumorigenicity of CSCs. Commonly expressed surface markers by the CSCs include CD44, CD133, and CD166. Al-Hajj, M., et al. Proc. Natl Acad. Sci. USA, 2003. 100(7): p. 3983-88; Collins, A. T., et al. Cancer Res., 2005. 65(23): p. 10946-51; Li, C., et al. Cancer Res., 2007. 67(3): p. 1030-37; Ma, S., et al. Gastroenterology, 2007. 132(7): p. 2542-56; Ricci-Vitiani, L., et al. Nature, 2007. 445(7123): p. 111-15; Singh, S. K., et al. Cancer Res., 2003. 63(18): p. 5821-28; and Bleau, A. M., et al., Neurosurg. Focus, 2008. 24(3-4): p. E28. Sorting tumor cells based primarily upon the differential expression of these surface marker(s) have accounted for the majority of the highly tumorigenic CSCs described to date. Therefore, these surface markers are validated for identification and isolation of CSCs from the cancer cell lines and from the bulk of tumor tissues.

By using aiRNA (asymmetric RNA duplexes), potent Stat3 selective silencing has been achieved in stemness-high cancer cells. This Stat3 silencing may lead to downregulation of cancer cell stemness, and/or inhibition of stemness-high cancer cell survival and self-renewal.

In some embodiments, the at least one compound of formula (I) is an inhibitor of CSC growth and survival. According to U.S. Pat. No. 8,877,803, the compound of formula (I) inhibits Stat3 pathway activity with a cellular IC₅₀ of ˜0.25 μM. The at least one compound of formula (I) may be synthesized according to U.S. Pat. No. 8,877,803, for example, Example 13. In some embodiments, the at least one compound of formula (I) is used in a method of treating cancers. According to PCT Patent Application No. PCT/US2014/033566, Example 6, the at least one compound of formula (I) was chosen to enter a clinical trial for patients with advanced cancers. The disclosures of U.S. Pat. No. 8,877,803 and PCT Patent Application No. PCT/US2014/033566 are incorporated herein by reference in their entireties.

We have surprisingly discovered that patients with higher expression levels of Stat3 show prolonged overall survival after treatment with at least one compound of formula (I) in clinical trials. Thus, the higher the level of pStat3 found in a cancer patient before treatment, at least in CRC patients, the higher the overall survival (OS) upon administering a treatment comprising a compound of formula (I).

We also have surprisingly discovered that a treatment combination of at least one compound of formula (I) with at least one paclitaxel compound results in anti-tumor activity in subjects with certain types of cancer that progressed on prior taxane treatment.

In some embodiments, disclosed herein are methods for treating cancer that had progressed on at least one prior taxane regimen comprising administering to a subject in need thereof:

a therapeutically effective amount of at least one compound of formula (I) chosen from compounds having formula (I):

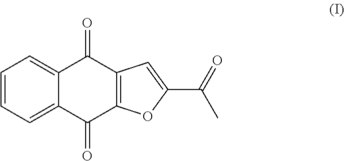 [SMILES: CC(=O)c1cc2c(o1)C(=O)c1ccccc1C2=O]

prodrugs, derivatives, pharmaceutically acceptable salts of any of the foregoing, and solvates of any of the foregoing, and

a therapeutically effective amount of at least one paclitaxel compound chosen from paclitaxel, pharmaceutically acceptable salt thereof, and solvates of any of the foregoing.

The at least one compound of formula (I) and the at least one paclitaxel compound may be administered to a subject simultaneously and/or sequentially.

The at least one compound of formula (I) may be administered daily in a single or a divided dose. The at least one paclitaxel compound may be administered weekly.

In some embodiments, disclosed herein are methods for resensitizing a subject to at least one prior therapy regimen comprising administering to a subject in need thereof:

a therapeutically effective amount of at least one compound of formula (I) chosen from compounds having formula (I):

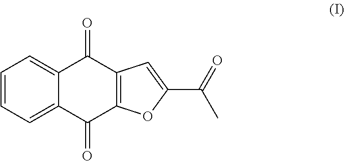 [SMILES: CC(=O)c1cc2c(o1)C(=O)c1ccccc1C2=O]

prodrugs, derivatives, pharmaceutically acceptable salts of any of the foregoing, and solvates of any of the foregoing. In some embodiments, the at least one prior therapy regimen is chosen from chemotherapy regimens. In some embodiments, the at least one prior therapy regimen chosen from taxane chemotherapy regimens. In some embodiments, disclosed herein are methods for resensitizing a subject to a taxane chemotherapy regimen comprising administering to a subject in need thereof:

a therapeutically effective amount of at least one compound of formula (I) chosen from compounds having formula (I):

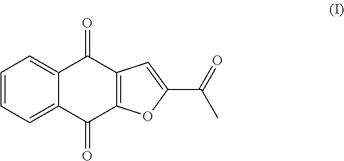 [SMILES: CC(=O)c1cc2c(o1)C(=O)c1ccccc1C2=O]

prodrugs, derivatives, pharmaceutically acceptable salts of any of the foregoing, and solvates of any of the foregoing.

In some embodiments, a kit is disclosed that comprises (1) at least one compound chosen from compounds having formula (I), prodrugs, derivatives, pharmaceutically acceptable salts of any of the foregoing, and solvates of any of the foregoing, and (2) at least one paclitaxel compound chosen from paclitaxel, pharmaceutically acceptable salts thereof, and solvates of any of the foregoing, together with instructions for administration and/or use.

Aspects and embodiments of the present disclosure are set forth or will be readily apparent from the following detailed description. It is to be understood that both the foregoing general description and the following detailed description are exemplary and explanatory only, and are not intended to be restrictive of the claims.

### BRIEF DESCRIPTION OF THE FIGURES

FIG. 1 shows the Stat3 pathway.

FIG. 2 shows the Stat3 pathway in cancer.

FIG. 3 shows the cancer stem cell specific and conventional cancer therapies.

FIG. 4 shows the initiation of relapse and metastases by cancer stem cells and cells with cancer stemness properties following treatment with conventional therapies.

FIG. 5 shows the effect of treatment of 2-acetylnaphtho[2,3-b]furan-4,9-dione on the protein levels of cancer stemness biomarkers p-Stat3 and β-catenin in human colon cancer xenograft Tumor (SW480) in nude mice.

FIG. 6 shows the effect of 2-acetylnaphtho[2,3-b]furan-4,9-dione, paclitaxel, and the combination of 2-acetylnaphtho[2,3-b]furan-4,9-dione together with paclitaxel in vitro on the protein levels of cancer stemness biomarkers pStat3 and β-catenin in pancreatic adenocarcinoma cancer stem cells (Panc-1).

FIG. 7 shows the greater than additive effect of the combination of 2-acetylnaphtho[2,3-b]furan-4,9-dione (“napabucasin”) together with paclitaxel on metabolic tumor volume in a human lung xenograft, A549, a mouse model.

FIG. 8 shows the percent change in target lesions (best response) in evaluable taxane naïve patients (N=19) with advanced pancreatic cancer treated with 2-acetylnaphtho[2,3-b]furan-4,9-dione and paclitaxel.

FIG. 9 shows the median progression free survival (PFS) and median overall survival (OS) of all patients (N=41), as well as each individual patient time on treatment and time without progression per RECIST, with advanced pancreatic cancer treated with 2-acetylnaphtho[2,3-b]furan-4,9-dione and paclitaxel.

FIG. 10A and FIG. 10B show the progression free survival (PFS) (FIG. 10A) and overall survival (OS) (FIG. 10B) of all taxane-naïve patients (N=23) with advanced pancreatic cancer treated with 2-acetylnaphtho[2,3-b]furan-4,9-dione and paclitaxel.

FIG. 11A and FIG. 11B show CT scans of a patient before and 16 weeks after receiving an exemplary treatment.

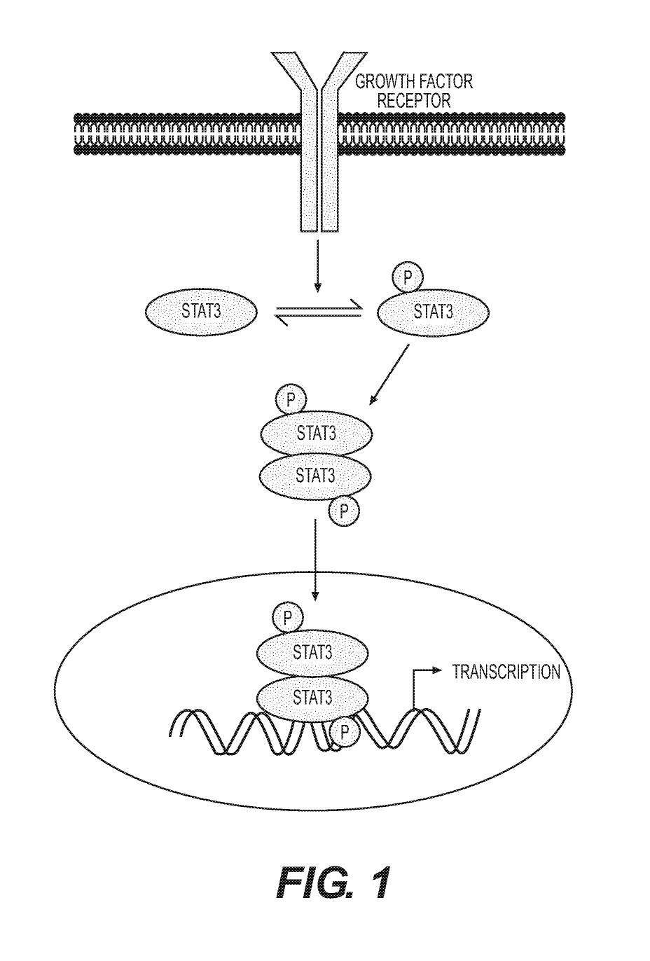

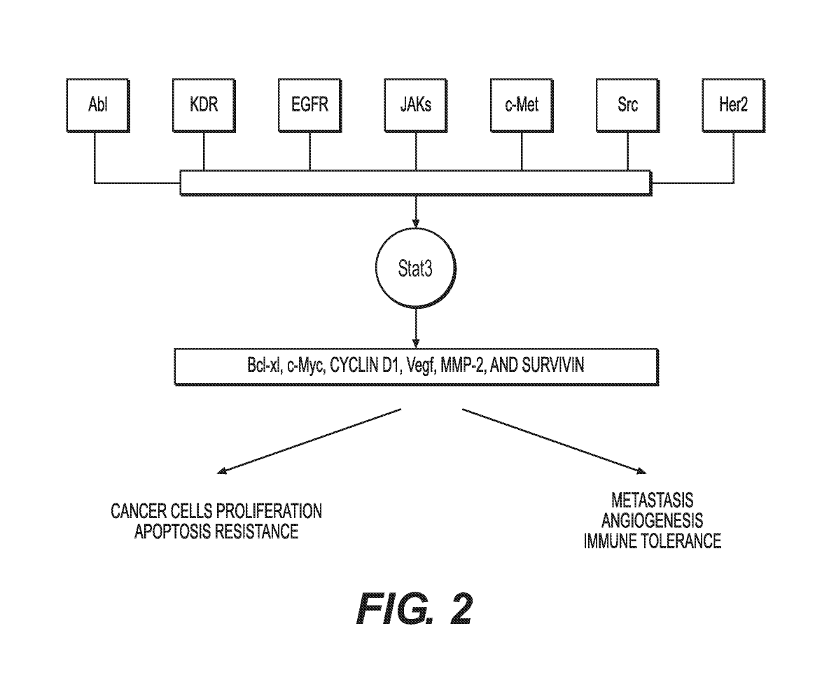

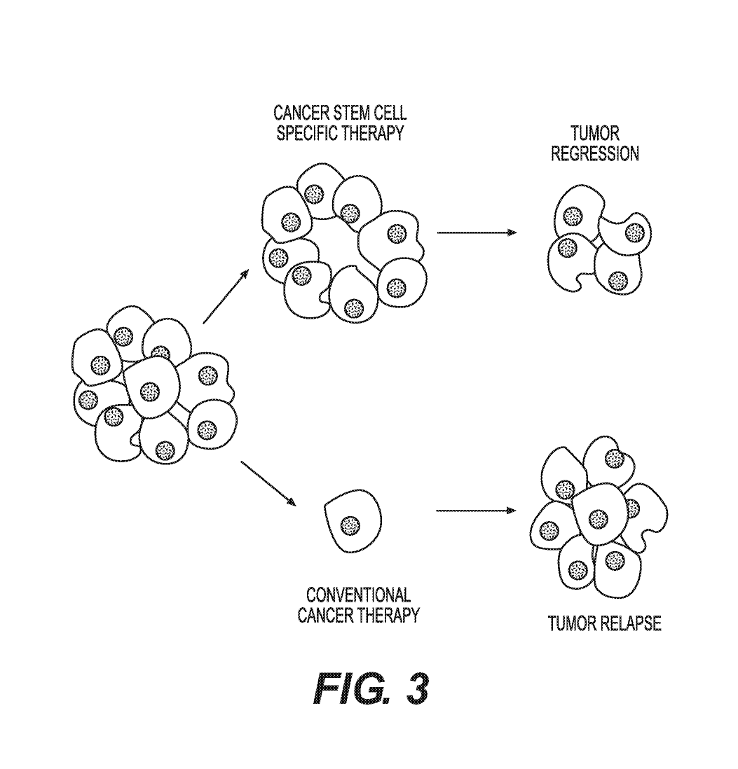

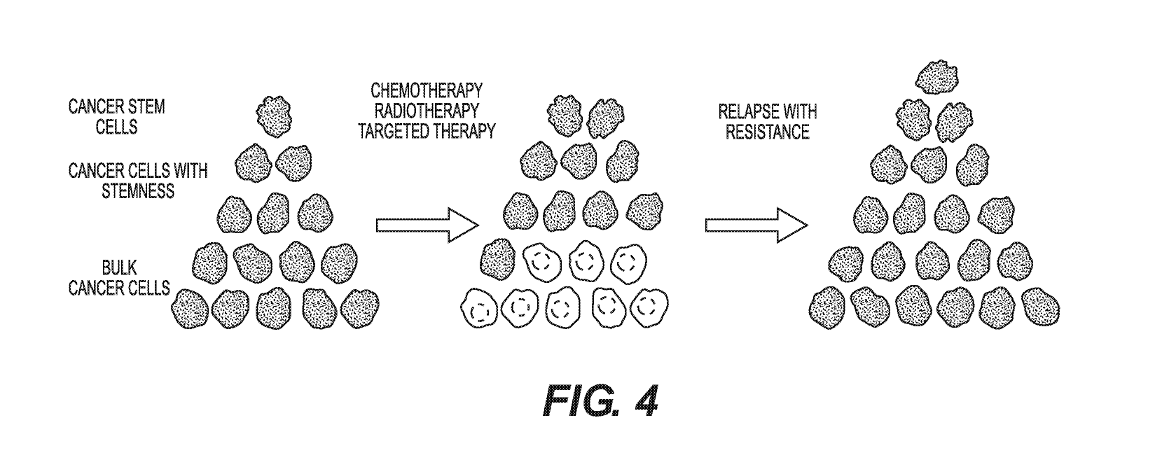

The following are definitions of terms used in the present specification. The initial definition provided for a group or term herein applies to that group or term throughout the present specification individually or as part of another group, unless otherwise indicated.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In some embodiments, the term “about” is used to modify a numerical value above and below the stated value by a variance of 10%. In some embodiments, the term “about” is used to modify a numerical value above and below the stated value by a variance of 5%. In some embodiments, the term “about” is used to modify a numerical value above and below the stated value by a variance of 1%.

The terms “administer,” “administering,” or “administration” are used herein in their broadest sense. These terms refer to any method of introducing to a subject a compound or pharmaceutical composition described herein and can include, for example, introducing the compound systemically, locally, or in situ to the subject. Thus, a compound of the present disclosure produced in a subject from a composition (whether or not it includes the compound) is encompassed in these terms. When these terms are used in connection with the term “systemic” or “systemically,” they generally refer to in vivo systemic absorption or accumulation of the compound or composition in the blood stream followed by distribution throughout the entire body.

The term “subject” generally refers to an organism to which a compound or pharmaceutical composition described herein can be administered. A subject can be a mammal or mammalian cell, including a human or human cell. The term also refers to an organism, which includes a cell or a donor or recipient of such cell. In various embodiments, the term “subject” refers to any animal (e.g., a mammal), including, but not limited to humans, mammals and non-mammals, such as non-human primates, mice, rabbits, sheep, dogs, cats, horses, cows, chickens, amphibians, and reptiles, which is to be the recipient of a compound or pharmaceutical composition described herein. Under some circumstances, the terms “subject” and “patient” are used interchangeably herein in reference to a human subject.

The terms “effective amount” and “therapeutically effective amount” refer to that amount of a compound or pharmaceutical composition described herein that is sufficient to effect the intended result including, but not limited to, disease treatment, as illustrated below. In some embodiments, the “therapeutically effective amount” is the amount that is effective for detectable killing or inhibition of the growth or spread of cancer cells, the size or number of tumors, and/or other measure of the level, stage, progression and/or severity of the cancer. In some embodiments, the “therapeutically effective amount” refers to the amount that is administered systemically, locally, or in situ (e.g., the amount of compound that is produced in situ in a subject). The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of cell migration. The specific dose may vary depending on, for example, the particular pharmaceutical composition, subject and their age and existing health conditions or risk for health conditions, the dosing regimen to be followed, the severity of the disease, whether it is administered in combination with other agents, timing of administration, the tissue to which it is administered, and the physical delivery system in which it is carried.

As used herein, the terms “treatment,” “treating,” “ameliorating,” and “encouraging” are used interchangeably herein. These terms refer to an approach for obtaining beneficial or desired results including, but not limited to, therapeutic benefit and/or prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject can still be afflicted with the underlying disorder. For prophylactic benefit, the pharmaceutical composition may be administered to a subject at risk of developing a particular disease, or to a subject reporting one or more of the physiological symptoms of a disease, even though a diagnosis of this disease may not have been made.

The term “cancer” in a subject refers to the presence of cells possessing characteristics typical of cancer-causing cells, such as uncontrolled proliferation, immortality, metastatic potential, rapid growth and proliferation rate, and certain morphological features. Often, cancer cells will be in the form of a tumor or mass, but such cells may exist alone within a subject, or may circulate in the blood stream as independent cells, such as leukemic or lymphoma cells. Examples of cancer as used herein include, but are not limited to, lung cancer, pancreatic cancer, bone cancer, skin cancer, head or neck cancer, cutaneous or intraocular melanoma, breast cancer, uterine cancer, ovarian cancer, peritoneal cancer, colon cancer, rectal cancer, colorectal adenocarcinoma, cancer of the anal region, stomach cancer, gastric cancer, gastrointestinal cancer, gastric adenocarcinoma, adrenocorticoid carcinoma, uterine cancer, carcinoma of the fallopian tubes, carcinoma of the endometrium, carcinoma of the vagina, carcinoma of the vulva, Hodgkin's Disease, esophageal cancer, gastroesophageal junction cancer, gastroesophageal adenocarcinoma, chondrosarcoma, cancer of the small intestine, cancer of the endocrine system, cancer of the thyroid gland, cancer of the parathyroid gland, cancer of the adrenal gland, sarcoma of soft tissue, Ewing's sarcoma, cancer of the urethra, cancer of the penis, prostate cancer, bladder cancer, testicular cancer, cancer of the ureter, carcinoma of the renal pelvis, mesothelioma, hepatocellular cancer, biliary cancer, kidney cancer, renal cell carcinoma, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. Some of the exemplified cancers are included in general terms and are included in this term. For example, urological cancer, a general term, includes bladder cancer, prostate cancer, kidney cancer, testicular cancer, and the like; and hepatobiliary cancer, another general term, includes liver cancers (itself a general term that includes hepatocellular carcinoma or cholangiocarcinoma), gallbladder cancer, biliary cancer, or pancreatic cancer. Both urological cancer and hepatobiliary cancer are contemplated by the present disclosure and included in the term “cancer.”

Also included within the term “cancer” is “solid tumor.” As used herein, the term “solid tumor” refers to those conditions, such as cancer, that form an abnormal tumor mass, such as sarcomas, carcinomas, and lymphomas. Examples of solid tumors include, but are not limited to, non-small cell lung cancer (NSCLC), neuroendocrine tumors, thyomas, fibrous tumors, metastatic colorectal cancer (mCRC), and the like. In some embodiments, the solid tumor disease is an adenocarcinoma, squamous cell carcinoma, large cell carcinoma, and the like.

In some embodiments, the cancer is chosen from gastric adenocarcinoma, gastroesophageal junction (GEJ) adenocarcinoma, non-small cell lung cancer (NSCLC), breast cancer, triple-negative breast cancer (TNBC; i.e., breast cancer that tests negative for estrogen receptors (ER−), progesterone receptors (PR−), and HER2 (HER2−)), ovarian cancer, platinum-resistant ovarian cancer (PROC), pancreatic adenocarcinoma, melanoma, small cell lung cancer, and cholangiocarcinoma. In some embodiments, the cancer is chosen from non-small cell lung cancer (NSCLC), breast cancer, triple-negative breast cancer (TNBC), ovarian cancer, platinum-resistant ovarian cancer (PROC), pancreatic adenocarcinoma, melanoma, small cell lung cancer, and cholangiocarcinoma. In some embodiments, the cancer is chosen from platinum-resistant ovarian cancer, triple-negative breast cancer, and non-small cell lung cancer. In some embodiments, the cancer is platinum-resistant ovarian cancer. In some embodiments, the cancer is triple-negative breast cancer. In some embodiments, the cancer is non-small cell lung cancer. In some embodiments, the cancer is not gastric adenocarcinoma. In some embodiments, the cancer is not gastroesophageal junction adenocarcinoma. In some embodiments, the cancer is not gastroesophageal junction adenocarcinoma or gastric adenocarcinoma.

The terms “progress,” “progressed,” and “progression” as used herein refer to at least one of the following: (1) a response to prior therapy (e.g., chemotherapy) of progressive disease (PD); (2) the appearance of one or more new lesions after treatment with prior therapy (e.g., chemotherapy); and (3) at least a 5% (e.g., 10%, 20%) increase in the sum of diameters of target lesions, taking as a reference the smallest sum on study (this includes the baseline sum if that is the smallest on study).

As used herein, “re-sensitizing” means making subjects who were previously resistant, non-responsive, or somewhat responsive to a prior therapy (e.g., chemotherapy) regimen sensitive, responsive, or more responsive to that prior therapy (e.g., chemotherapy) regimen.

As used herein, the term “at least one compound of formula (I)” means a compound chosen from compounds having formula (I)

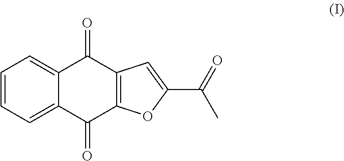 [SMILES: CC(=O)c1cc2c(o1)C(=O)c1ccccc1C2=O]

prodrugs, derivatives, pharmaceutically acceptable salts of any of the foregoing, and solvates of any of the foregoing. In some embodiments, prodrugs and derivatives of compounds having formula (I) are Stat3 inhibitors. Non-limiting examples of prodrugs of compounds having formula (I) are the phosphoric ester and phosphoric diester described in U.S. pre-grant Publication No. 2012/0252763 as compound numbers 4011 and 4012 and also suitable compounds described in in U.S. Pat. No. 9,150,530. Non-limiting examples of derivatives of compounds having formula (I) include the derivatives disclosed in U.S. Pat. No. 8,977,803. The disclosures of U.S. pre-grant Publication No. 2012/0252763 and U.S. Pat. Nos. 9,150,530 and 8,977,803 are incorporated herein by reference in their entireties.

Compounds having formula (I), shown below,

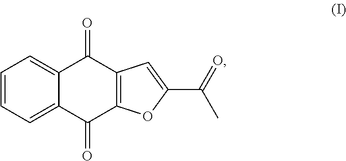 [SMILES: CC(=O)c1cc2c(o1)C(=O)c1ccccc1C2=O]

may also be known as 2-acetylnaphtho[2,3-b]furan-4,9-dione, napabucasin, or BBI608 and include tautomers thereof.

Suitable methods of preparing 2-acetylnaphtho[2,3-b]furan-4,9-dione, including its crystalline forms and additional cancer stemness inhibitors, are described in the co-owned PCT applications published as WO 2009/036099, WO 2009/036101, WO 2011/116398, WO 2011/116399, and WO 2014/169078; the contents of each application is incorporated herein by reference.

The term “salt(s),” as used herein, includes acidic and/or basic salts formed with inorganic and/or organic acids and bases. As used herein, the term “pharmaceutically acceptable salt” refers to those salts which are, within the scope of sound medical judgment, suitable for use in contact with the tissues of subjects without undue toxicity, irritation, allergic response and/or the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19.

Pharmaceutically acceptable salts may be formed with inorganic or organic acids. Non-limiting examples of suitable inorganic acids include hydrochloric acid, hydrobromic acid, phosphoric acid, sulfuric acid, and perchloric acid. Non-limiting examples of suitable organic acids include acetic acid, oxalic acid, maleic acid, tartaric acid, citric acid, succinic acid, and malonic acid. Other non-limiting examples of suitable pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hem isulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid.

Salts may be prepared in situ during the isolation and purification of the disclosed compound, or separately, such as by reacting the compound with a suitable base or acid, respectively. Non-limiting examples of pharmaceutically acceptable salts derived from bases include alkali metal, alkaline earth metal, ammonium and N⁺(C₁₋₄alkyl)₄ salts. Non-limiting examples of suitable alkali or alkaline earth metal salts include sodium, lithium, potassium, calcium, magnesium, iron, zinc, copper, manganese, and aluminum salts. Further non-limiting examples of suitable pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Non-limiting examples of suitable organic bases from which salts may be derived include primary amines, secondary amines, tertiary amines, substituted amines including naturally occurring substituted amines, cyclic amines, and basic ion exchange resins, such as isopropylamine, trimethylamine, diethylamine, triethylamine, tripropylamine, and ethanolamine. In some embodiments, pharmaceutically acceptable base addition salts can be chosen from ammonium, potassium, sodium, calcium, and magnesium salts.

The term “solvate” represents an aggregate that comprises one or more molecules of a compound of the present disclosure with one or more molecules of a solvent or solvents. Solvates of the compounds of the present disclosure include, for example, hydrates.

In some embodiments, the at least one paclitaxel compound is administered once weekly as an IV infusion. In some embodiments, the at least one paclitaxel compound is administered at about 80 mg/m² weekly for 3 out of every 4 weeks.

The at least one compound disclosed herein may be in the form of a pharmaceutical composition. In some embodiments, the pharmaceutical compositions may comprise the at least one compound of formula (I) and at least one pharmaceutically acceptable carrier. In some embodiments, the pharmaceutical compositions may comprise one or more compounds and at least one pharmaceutically acceptable carrier, where the one or more compounds are capable of being converted into the at least one compound of formula (I) in a subject (i.e., a prodrug).

The term “carrier” as used herein means a pharmaceutically acceptable material, composition or vehicle, such as, for example, a liquid or solid filler, diluent, excipient, solvent or encapsulating material involved in or capable of carrying or transporting the subject pharmaceutical compound from one organ, or portion of the body, to another organ, or portion of the body. Each carrier must be “acceptable” in the sense of being compatible with the other ingredients of the formulation and not injurious to the patient. Non-limiting examples of pharmaceutically acceptable carriers, carriers, and/or diluents include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers, and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

In some embodiments, the at least one compound may be administered in an amount ranging from about 160 to about 1500 mg. In some embodiments, the at least one compound may be administered in an amount ranging from about 160 to about 1000 mg. In some embodiments, the at least one compound may be administered in an amount ranging from about 300 mg to about 700 mg. In some embodiments, the at least one compound may be administered in an amount ranging from about 700 mg to about 1200 mg. In some embodiments, the at least one compound may be administered in an amount ranging from about 800 mg to about 1100 mg. In some embodiments, the at least one compound may be administered in an amount ranging from about 850 mg to about 1050 mg. In some embodiments, the at least one compound may be administered in an amount ranging from about 960 mg to about 1000 mg. In some embodiments, the total amount of the at least one compound is administered once daily. In some embodiments, the at least one compound is administered in a dose of about 480 mg daily. In some embodiments, the at least one compound is administered in administered in a dose of about 960 mg daily. In some embodiments, the at least one compound is administered in a dose of about 1000 mg daily. In some embodiments, the total amount of the at least one compound is administered in divided doses more than once daily, such as twice daily (BID) or more often. In some embodiments, the at least one compound may be administered in an amount ranging from about 80 to about 750 mg twice daily. In some embodiments, the at least one compound may be administered in an amount ranging from about 80 to about 500 mg twice daily. In some embodiments, the at least one compound is administered in a dose of about 240 mg twice daily. In some embodiments, the at least one compound is administered in a dose of about 480 mg twice daily. In some embodiments, the at least one compound is administered in a dose of about 500 mg twice daily.

Pharmaceutical compositions disclosed herein that are suitable for oral administration may be in the form of capsules, cachets, pills, tablets, lozenges (using a flavored basis, usually sucrose and acacia or tragacanth), powders, granules, a solution in an aqueous or non-aqueous liquid, a suspension in an aqueous or non-aqueous liquid, an oil-in-water emulsion, a water-in-oil emulsion, an elixir, a syrup, pastilles (using an inert base, such as gelatin, glycerin, sucrose, and/or acacia) and/or mouthwashes, each containing a predetermined amount of the at least one compound of the present disclosure.

A pharmaceutical composition disclosed herein may be administered as a bolus, electuary, or paste.

Solid dosage forms for oral administration (capsules, tablets, pills, dragees, powders, granules and the like) may be mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polypropylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type also may be employed as fillers in soft and hard-filled gelatin capsules using such excipients as lactose or milk sugars, as well as high molecular weight polyethylene glycols and the like.

Liquid dosage forms for oral administration may include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxypropyl-β-cyclodextrin, may be used to solubilize compounds.

The pharmaceutical compositions also may include adjuvants such as wetting agents, emulsifying and suspending agents, sweetening, flavoring, coloring, perfuming and preservative agents. Suspensions, in addition to the compounds according to the disclosure, may contain suspending agents as, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions disclosed herein, for rectal or vaginal administration may be presented as a suppository, which may be prepared by mixing one or more compounds according to the present disclosure, with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the compounds of the present disclosure. Pharmaceutical compositions which are suitable for vaginal administration also may include pessaries, tampons, creams, gels, pastes, foams or spray formulations containing carriers that are known in the art to be appropriate.

Dosage forms for the topical or transdermal administration of a pharmaceutical composition or pharmaceutical tablet of the present disclosure may include powders, sprays, ointments, pastes, creams, lotions, gels, solutions, patches and inhalants. The pharmaceutical composition or pharmaceutical tablet may be mixed under sterile conditions with a pharmaceutically acceptable carrier, and with any preservatives, buffers, or propellants which may be required.

The ointments, pastes, creams and gels may contain, in addition to the pharmaceutical composition or pharmaceutical tablet of the present disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a pharmaceutical composition or a pharmaceutical tablet of the present disclosure, excipients such as lactose, talc, silicic acid, aluminum hydroxide, calcium silicates and polyamide powder, or mixtures of these substances. Additionally, sprays may contain customary propellants, such as chlorofluorohydrocarbons and volatile unsubstituted hydrocarbons, such as butane and propane.

Ophthalmic formulations, eye ointments, powders, solutions and the like, are also contemplated as being within the scope of the present disclosure.

Compositions suitable for parenteral administration may comprise at least one more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In various embodiments, a composition described herein includes at least one compound chosen from compounds of formula (I) and pharmaceutically acceptable salts and solvates thereof and one or more surfactants. In some embodiments, the surfactant is sodium lauryl sulfate (SLS), sodium dodecyl sulfate (SDS), or one or more polyoxylglycerides. For example, the polyoxyglyceride can be lauroyl polyoxylglycerides (sometimes referred to as Gelucire™) or linoleoyl polyoxylglycerides (sometimes referred to as Labrafil™). Examples of such compositions are shown in PCT Patent Application No. PCT/US2014/033566, the contents of which are incorporated herein in its entirety.

As noted above, the methods disclosed herein may treat at least one disorder related to aberrant Stat3 pathway activity in a subject. Aberrant Stat3 pathway activity can be identified by expression of phosphorylated Stat3 (“pStat3”) or its surrogate upstream or downstream regulators.

The Stat3 pathway can be activated in response to cytokines, for example, IL-6, or by one or more tyrosine kinases, for example, EGFR, JAKs, Abl, KDR, c-Met, Src, and Her2. The downstream effectors of Stat3 include but are not limited to Bcl-xl, c-Myc, cyclinD1, Vegf, MMP-2, and survivin. The Stat3 pathway has been found to be aberrantly active in a wide variety of cancers, as shown in Table 1. Persistently active Stat3 pathway may occur in more than half of breast and lung cancers, hepatocellular carcinomas, multiple myelomas and in more than 95% of head and neck cancers. Blocking the Stat3 pathway causes cancer cell-growth arrest, apoptosis, and reduction of metastasis frequency in vitro and/or in vivo.

TABLE 1
DISEASES

| ONCOLOGY | Solid | Breast Cancer (Watson, C. J. and W. R. |
|---|---|---|
| DISEASES | tumors | Miller. Br. J. Cancer, 1995. 71(4): p. |
| | | 840-44) |
| | | Head and Neck Cancer (SCCHN) (Song, J. |
| | | I. and J. R. Grandis. Oncogene, 2000. |
| | | 19(21): p. 2489-95) |
| | | Lung Cancer (Song, L., et al. Oncogene, |
| | | 2003. 22(27): p. 4150-65) |
| | | Ovarian Cancer (Savarese, T. M., et al. |
| | | Cytokine, 2002. 17(6): p. 324-34) |
| | | Pancreatic Cancer (Toyonaga, T., et al. |
| | | Cancer Lett., 2003. 201(1): p. 107-16) |
| | | Colorectal carcinoma (Corvinus, F. M., |
| | | et al. Neoplasia, 2005. 7(6): p. 545-55) |
| | | Prostate Cancer (Gao, B., et al. FEBS |
| | | Lett., 2001. 488(3): p. 179-84) |
| | | Renal Cell carcinoma (Buettner, R., et al. |
| | | Clin. Cancer Res., 2002. 8(4): p. 945-54) |
| | | Melanoma (Carson, W. E. Clin. Cancer |
| | | Res., 1998. 4(9): p. 2219-28) |
| | | Hepatocellular carcinomas (Darnell, J. E. |
| | | Nat. Med., 2005. 11(6): p. 595-96) |
| | | Cervical Cancer (Chen, C. L., et al. Br. J. |
| | | Cancer, 2007. 96(4): p. 591-99) |
| | | Endometrial Cancer (Chen, C. L., et al. |
| | | Br. J. Cancer, 2007. 96(4): p. 591-99) |
| | | Sarcomas (Lai, R., et al. J. Pathol., 2006. |
| | | 208(5): p. 624-32; and) |
| | | Brain Tumors (Punjabi, A. S., et al. J. |
| | | Virol., 2007. 81(5): p. 2449-58) |
| | | Gastric Cancers (Kanda, N., et al. |
| | | Oncogene, 2004. 23(28): p. 4921-29) |
| | Hemato- | Multiple Myeloma (Puthier, D., et al. |
| | logic | Eur. J. Immunol., 1999. 29(12): p. |
| | Tumors | 3945-50) |

| | |
|---|---|
| Leukemia | HTLV-1-dependent Leukemia |
| | (Migone, T. S., et al. Science, |
| | 1995. 269(5220): p. 79-81) |
| | Chronic Myelogenous |
| | Leukemia (Buettner, R., et al. |
| | Clin. Cancer Res., 2002. 8(4): |
| | p. 945-54) |
| | Acute Myelogenous Leukemia |
| | (Spiekermann, K., et al. Eur. |
| | J. Haematol., 2001. 67(2): p. |
| | 63-71) |
| | Large Granular Lymphocyte |
| | Leukemia (Epling-Burnette, |
| | P. K., et al. J. Clin. |
| | Invest., 2001. 107(3): p. |
| | 351-62) |
| Lymphomas | EBV-related/Burkitt's |
| | (Weber-Nordt, R. M., et al. |
| | Blood, 1996. 88(3): p. |
| | 809-16) |
| | Mycosis Fungoides (Buettner, |
| | R., et al. Clin. Cancer Res., |
| | 2002. 8(4): p. 945-54) |
| | HSV Saimiri-dependent (T- |
| | cell) (Buettner, R., et al. Clin. |
| | Cancer Res., 2002. 8(4): p. |
| | 945-54) |
| | Cutaneous T-cell Lymphoma |
| | (Sommer, V. H., et al. |
| | Leukemia, 2004. 18(7): p. |
| | 1288-95) |
| | Hodgkin's Diseases (Buettner, |
| | R., et al. Clin. Cancer Res., |
| | 2002. 8(4): p. 945-54) |
| | Anaplastic Large-cell |
| | Lymphoma (Lai, R., et al. |
| | Am. J. Pathol., 2004. 164(6): |
| | p. 2251-58) |

In some embodiments, the at least one disorder may be chosen from cancers related to aberrant Stat3 pathway activity, such as gastric carcinoma, gastroesophageal junction adenocarcinoma, colorectal carcinoma, pancreatic cancer, breast cancer, ovarian cancer, fallopian tube cancer, peritoneal cancer, head and neck cancer, melanoma, cholangiocarcinoma, and lung cancer.

Recent studies have disclosed that CSCs are able to regenerate tumors. These CSCs are disclosed to be functionally linked with continued malignant growth, cancer metastasis, recurrence, and cancer drug resistance. CSCs and their differentiated progeny appear to have markedly different biologic characteristics. They persist in tumors as a distinct, but rare population. Conventional cancer drug screenings depend on measurement of the amount of tumor mass and, therefore, may not identify drugs that act specifically on the CSCs. In fact, CSCs have been disclosed to be resistant to standard chemotherapies and are enriched after standard chemotherapy treatments, which can result in refractory cancer and recurrence. CSCs have also been demonstrated to be resistant to radiotherapy. Baumann, M., et al. Nat. Rev. Cancer, 2008. 8(7): p. 545-54. The reported cancer types in which CSCs have been isolated include breast cancer, head cancer, neck cancer, lung cancer, ovarian cancer, pancreatic cancer, colorectal carcinoma, prostate cancer, melanoma, multiple myeloma, Kaposi sarcoma, Ewing's sarcoma, liver cancer, medulloblastoma, brain tumors, and leukemia. Stat3 has been identified as a CSCsurvival and self-renewal factor. Therefore, Stat3 inhibitors may kill CSCs and/or may inhibit CSC self-renewal. According to some embodiments, cancer stem cell or cancer stem cells refer to a minute population of CSCs that have self-renewal capability and are tumorigenic.

Disclosed herein are methods of inhibiting, reducing, and/or diminishing CSC survival and/or self-renewal comprising administering a therapeutically effective amount of at least one pharmaceutical composition comprising at least one compound of formula (I) in combination with a therapeutically effective amount of at least one paclitaxel compound chosen from paclitaxel, pharmaceutically acceptable salts thereof, and solvates of any of the foregoing. Also disclosed herein are methods of inhibiting, reducing, and/or diminishing CSC survival and/or self-renewal comprising administering a therapeutically effective amount of at least one compound of formula (I) in combination with a therapeutically effective amount of at least one paclitaxel compound chosen from paclitaxel, pharmaceutically acceptable salts thereof, and solvates of any of the foregoing.

Also disclosed herein are methods of treating at least one cancer that is refractory to conventional chemotherapies and/or targeted therapies in a subject comprising administering a therapeutically effective amount of at least one compound of formula (I) a in combination with a therapeutically effective amount of at least one paclitaxel compound chosen from paclitaxel, pharmaceutically acceptable salts thereof, and solvates of any of the foregoing. In some embodiments, the at least one compound is included in a pharmaceutical composition.

Disclosed herein are methods of treating recurrent cancer in a subject that has failed surgery, oncology therapy (e.g., chemotherapy), and/or radiation therapy, comprising administering a therapeutically effective amount of at least one compound of formula (I) in combination with a therapeutically effective amount of at least one paclitaxel compound chosen from paclitaxel, pharmaceutically acceptable salts thereof, and solvates of any of the foregoing. In various embodiments, the at least one compound of formula (I) is included in a pharmaceutical composition.

Also disclosed herein are methods of treating or preventing cancer metastasis in a subject, comprising administering a therapeutically effective amount of at least one compound of formula (I) in combination with a therapeutically effective amount of at least one paclitaxel compound chosen from paclitaxel, pharmaceutically acceptable salts thereof, and solvates of any of the foregoing. In various embodiments, the at least one compound of formula (I) is included in a pharmaceutical composition.

Disclosed herein are methods of treating cancer in a subject comprising administering a therapeutically effective amount of at least one compound of formula (I) in combination with a therapeutically effective amount of at least one paclitaxel compound chosen from paclitaxel, pharmaceutically acceptable salts thereof, and solvates of any of the foregoing. In various embodiments, the at least one compound of formula (I) is included in a pharmaceutical composition.

In some embodiments, the cancer may be chosen from gastric and gastroesophageal adenocarcinoma, advanced gastric and gastroesophageal junction adenocarcinoma, colorectal adenocarcinoma, breast cancer, ovarian cancer, head and neck cancer, melanoma, lung cancer, cholangiocarcinoma, and pancreatic cancer. In some embodiments, the cancer may be chosen from breast cancer, ovarian cancer, head and neck cancer, melanoma, lung cancer, cholangiocarcinoma, and pancreatic cancer. In some embodiments, the cancer is not gastric or gastroesophageal junction adenocarcinoma. In some embodiments, the cancer is metastatic pancreatic adenocarcinoma. In some embodiments, the cancer is advanced triple negative breast cancer. In some embodiments, the cancer is advanced non-small cell lung cancer. In some embodiments, the cancer is platinum resistant ovarian cancer. In some embodiments, the cancer is cholangiocarcinoma.

In some embodiments, the cancer may be advanced. In some embodiments, the cancer may be refractory. In some embodiments, the cancer may be recurrent. In some embodiments, the cancer may be metastatic. In some embodiments, the cancer may be associated with overexpression of Stat3. In some embodiments, the cancer may be associated with nuclearp-βcatenin localization.

### EXAMPLES

The methods disclosed herein comprise administering to a subject in need thereof a therapeutically effective amount of at least one paclitaxel compound chosen from paclitaxel, pharmaceutically acceptable salts thereof, and solvates of any of the foregoing and at least one compound of formula (I).

### Example 1

The effects of 2-acetylnaphtho[2,3-b]furan-4,9-dione, a compound of formula (I), on cancer stem cell markers was examined in cancer xenograft models with and without paclitaxel.

Human cancer cells were subcutaneously implanted into the right flank of 5-7 weeks old female athymic nude mice. When tumor size reached 200 mm³, animals were treated with 2-acetylnaphtho[2,3-b]furan-4,9-dione, for example, at 50 mg/kg (BID) by oral gavage (n=3/group), paclitaxel, or a combination of 2-acetylnaphtho[2,3-b]furan-4,9-dione and paclitaxel. Tumors were harvested at 24 hours after first dosing.

The harvested tissues were fixed in 3.7% neutral buffered formaldehyde at 4° C. for overnight. The paraffin was embedded, cut to about 5 microns, and affixed onto positively-charged slides. After being baked and de-paraffinized, the slides with tumor or control tissues were incubated in 10 mM sodium citrate (pH 6.0) for 10 minutes. After antigen retrieval, slides were probed with primary antibodies P-STAT3 (rabbit, Cell Signaling, 1:100), β-Catenine (mouse, Santa Cruz, 1:400) at 4° C. overnight, and then Alexa Fluor fluorescent dyes-conjugated secondary antibodies (1:500, Invitrogen). After mounting, the slides with ProLong mounting medium with DAPI (Invitrogen) were examined under a Zeiss fluorescence microscope with 20× objective, and analyzed with Zen software.

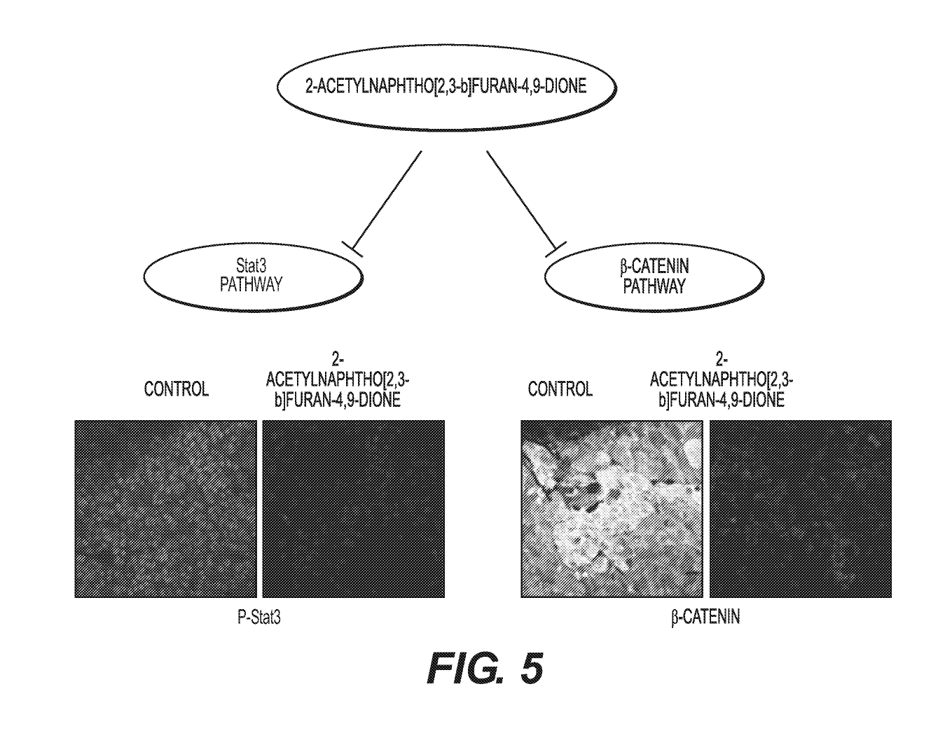

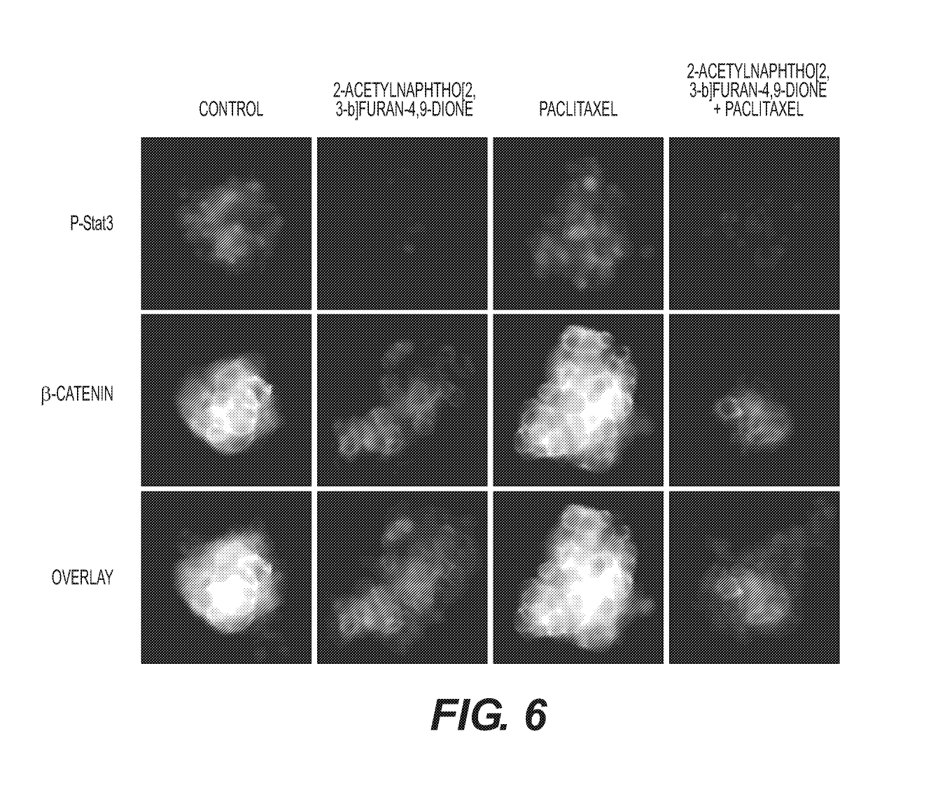

As shown in FIG. 5, 2-acetylnaphtho[2,3-b]furan-4,9-dione alone dramatically reduced expression of both the p-Stat3 and β-catenin stem cell markers. In contrast, as shown in FIG. 6, paclitaxel alone resulted in enhanced staining for stem cell markers, which was attenuated by the addition of 2-acetylnaphtho[2,3-b]furan-4,9-dione.

### Example 2

The effects of 2-acetylnaphtho[2,3-b]furan-4,9-dione was examined in a human lung cancer xenograft (A549) model. Human lung cancer cells were implanted in mice and the resulting tumors were allowed to grow to a pre-determined size. Mice were treated orally with vehicle, 2-acetylnaphtho[2,3-b]furan-4,9-dione (100 mg/kg, daily, oral), paclitaxel (10.0 mg/kg, q3d, iv), or the combination of 2-acetylnaphtho[2,3-b]furan-4,9-dione (100 mg/kg, daily, oral) and paclitaxel (10.0 mg/kg, q3d, iv). The tumor sizes were monitored.

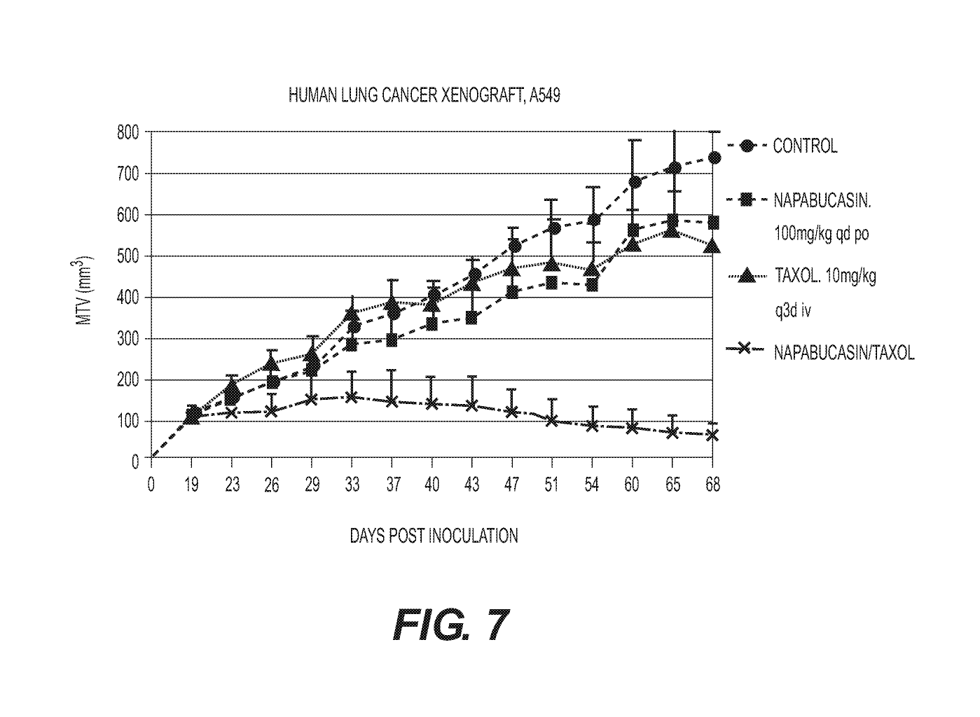

As shown in FIG. 7, both 2-acetylnaphtho[2,3-b]furan-4,9-dione (“napabucasin”) alone and paclitaxel (“taxon”) alone reduced metabolic tumor volume relative to control. As shown in FIG. 7, the combination of 2-acetylnaphtho[2,3-b]furan-4,9-dione with paclitaxel had a greater effect on metabolic tumor volume than the added effects of both agents alone. Thus, the combination of 2-acetylnaphtho[2,3-b]furan-4,9-dione and paclitaxel had a surprising effect on metabolic tumor volume in this human lung cancer xenograft model.

### Example 3

The effects of 2-acetylnaphtho[2,3-b]furan-4,9-dione, a compound of formula (I), in combination with paclitaxel in patients with heavily pretreated metastatic pancreatic adenocarcinoma were studied in a phase Ib/II extension study to assess the safety, tolerability, and preliminary anti-cancer activity of the combination disclosed herein.

In the open label phase lb dose-escalation study, the safety, tolerability and recommended phase 2 dose (RP2D) of 2-acetylnaphtho[2,3-b]furan-4,9-dione in combination with paclitaxel were assessed in adult patients with advanced solid tumors.

The phase II clinical study enrolled patients to disease-specific cohorts to determine the preliminary anti-cancer activity of 2-acetylnaphtho[2,3-b]furan-4,9-dione in combination with paclitaxel.

A sample size of 40 in each cohort set the bounds of the 90% Cl at ±10% to 14%, assuming a disease control rate (DCR) of 60% to 80%.

In total, 41 patients with heavily pre-treated pancreatic adenocarcinoma aged 38-82 were enrolled in the phase Ib/II extension study (see Table 2). As shown in Table 3, these patients received a median of 2 prior lines of treatment including FOLFIRINOX (71%), gemcitabine/nab-paclitaxel (44%), or both (37%). Most patients had failed the gemcitabine/nab-paclitaxel and/or FOLFIRINOX treatment. Overall, prior therapy included gemcitabine (90%), a thymidylate synthetase inhibitor (e.g., fluorouracil (5-FU) and capecitabine) (81%), platinum (76%), irinotecan (73%), and taxane (44%).

TABLE 2
Demographics (N = 41)

| Age | | |
|---|---|---|
| Median | 65 | yrs |
| Range | 38-82 | yrs |
| Gender | N | % |
| Female | 19 | 46% |
| Male | 22 | 54% |
| Karnofsky | N | % |
| 90% | 20 | 49% |
| 80% | 15 | 37% |
| 70% | 4 | 10% |
| ECOG | N | % |
| 1 | 2 | 5% |
| Race | N | % |
| Caucasian | 33 | 80% |
| Black | 5 | 12% |
| Asian | 1 | 2% |
| Other | 2 | 5% |

TABLE 3
Prior Treatments (N = 41)

| | N | % |
|---|---|---|
| Prior Surgery | | |
| Any surgery for cancer | 22 | 54% |
| Whipple procedure | 12 | 29% |
| Other pancreatic resection | 5 | 12% |
| Other resection | 10 | 24% |
| Prior Regimen | | |
| 1 prior | 14 | 34% |
| 2 prior | 16 | 39% |
| ≥3 prior | 11 | 27% |
| Prior Taxane | | |
| No | 23 | 56% |
| Yes | 18 | 44% |
| Prior Treatment for Cancer | | |
| FOLFIRINOX | 29 | 71% |
| Gemcitabine + Nab-paclitaxel | 18 | 44% |
| FOLFIRINOX & Gem + Nab-paclitaxel (both) | 15 | 37% |
| Any Gemcitabine | 37 | 90% |
| Any 5-FU | 33 | 80% |
| Gemcitabine plus 5-FU | 29 | 71% |
| Platinum | 31 | 76% |
| Irinotecan | 30 | 73% |
| Gemcitabine as only therapy | 6 | 15% |
| Erlotinib | 2 | 5% |

The 31 evaluable patients received 2-acetylnaphtho[2,3-b]furan-4,9-dione in combination with paclitaxel. Patients received oral administration of 2-acetylnaphtho[2,3-b]furan-4,9-dione twice daily together with paclitaxel. Specifically, 2-acetylnaphtho[2,3-b]furan-4,9-dione was administered at a starting dose of 480 mg or 500 mg BID in combination with paclitaxel at 80 mg/m² administered weekly as an IV infusion over one hour for 3 out every 4 weeks. Objective tumor response was assessed every 8 weeks using Response Evaluation Criteria In Solid Tumors (RECIST 1.1).

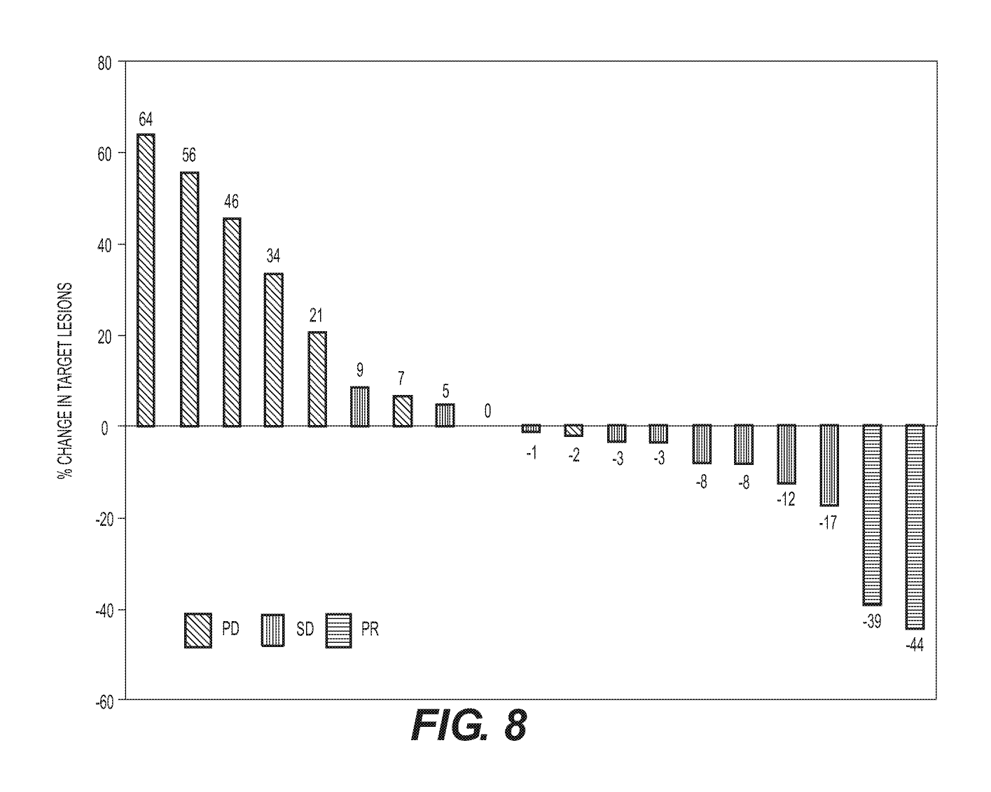

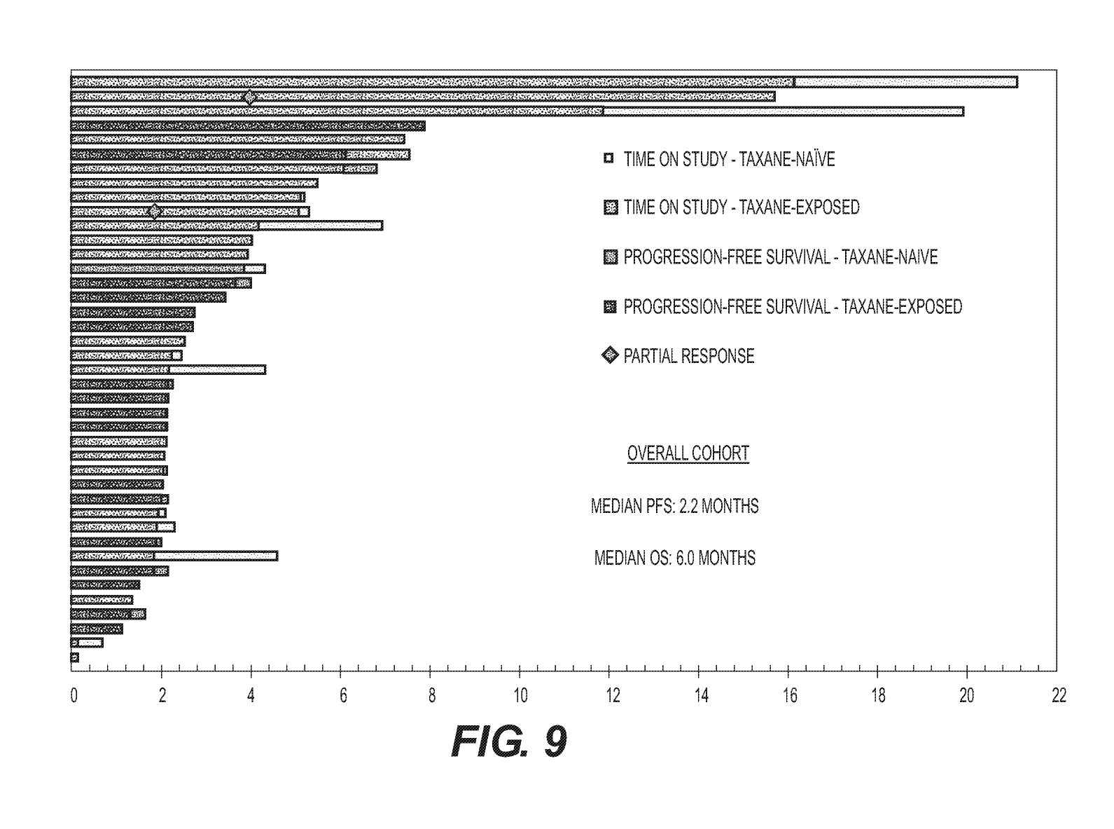

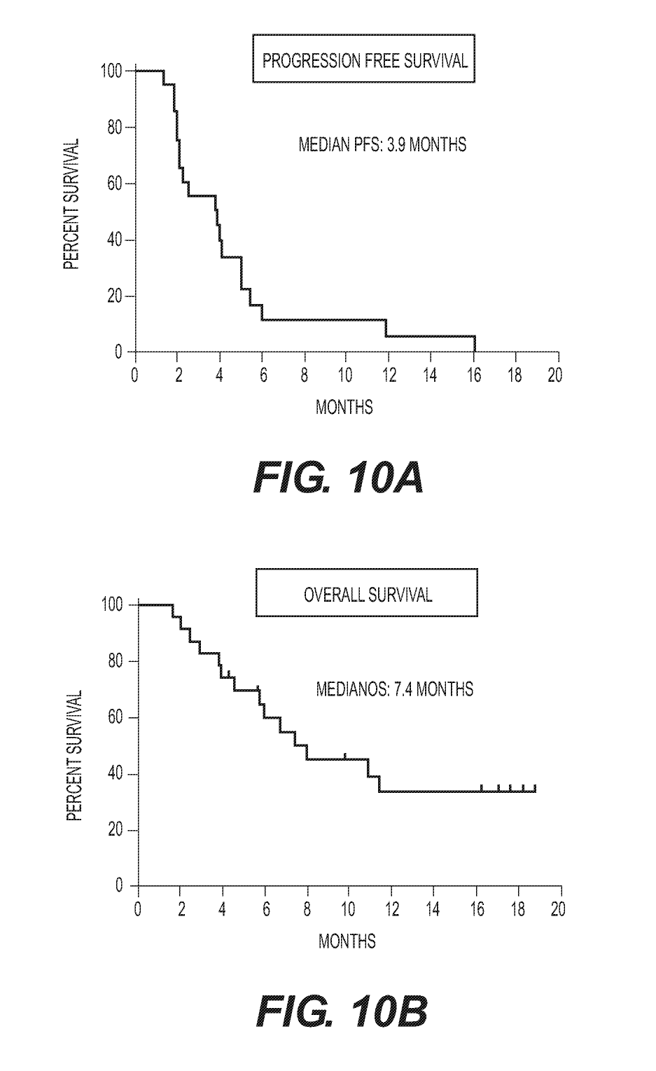

Anti-cancer activity was observed in patients with heavily pretreated metastatic pancreatic adenocarcinoma (see FIG. 8, FIG. 9, and FIG. 10). In addition, many patients continued on the treatment well after progression “per RECIST.” For example, as shown in Table 4, the evaluable patients (N=31) had a 6% response rate (partial response (PR)+complete response (CR)). This same group had a 48% disease control rate (stable disease (SD)+(PR)+(CR)). The evaluable taxane-naïve patients (N=19) had an 11% response rate, 63% disease control rate (see also FIG. 8), and 4 patients experienced >50% decrease in CA19-9. Additionally, 16% of the evaluable taxane-naïve patients were progression free at 24 weeks. Overall (intention-to-treat (ITT), N=41), the median progression-free survival (mPFS) was about 10 weeks and median overall survival (mOS) was 24 weeks (see, e.g., FIG. 9). For the taxane-naïve patients (ITT, N=23), mPFS was about 16 weeks and mOS was about 30 weeks (see, e.g., Table 5 and FIG. 10). In comparison, the mOS for patients with advanced, previously treated pancreatic adenocarcinoma treated with weekly paclitaxel alone was previously reported as being about 17.5 weeks (Oettle et al, Anticancer Drugs, 11:635-638 (2000)).

TABLE 4
All Evaluated Patients (N = 31*)

| | | Average | Objective | Disease | Disease |
|---|---|---|---|---|---|
| | | Lines | Response | Control | Control ≥24 |
| | | Prior | Rate | Rate | weeks |
| Cohort | N | Therapy | ORR, % | DCR, % | DCR24, % |
| Overall | 31 | 2.0 | 6% | 48% | 16% |
| Taxane Naïve | 19 | 1.6 | 11% | 63% | 16% |

*8 patients had early disease-related symptoms and 2 patients withdrew consent prior to on-study scan

TABLE 5
All Patients - Intent to Treat (N = 41)

| | | Average | Objective | Disease | Disease |
|---|---|---|---|---|---|
| | | Lines | Response | Control | Control ≥24 |
| | | Prior | Rate | Rate | weeks |
| Cohort | N | Therapy | ORR, % | DCR, % | DCR24, % |
| Overall | 41 | 2.0 | 5% | 37% | 12% |
| Taxane Naïve | 23 | 1.6 | 9% | 52% | 13% |

This study demonstrates that 2-acetylnaphtho[2,3-b]furan-4,9-dione (480 mg or 500 mg BID) combined with weekly paclitaxel was safe, tolerable, and effectively promoted anti-tumor activity in patients with advanced pancreatic adenocarcinoma including: objective responses, CA 19-9 improvements, prolonged disease control, and surprising progression-free and overall survival.

This study further demonstrates that 2-acetylnaphtho[2,3-b]furan-4,9-dione (480 mg or 500 mg BID) combined with weekly paclitaxel effectively promoted anti-tumor activity in taxane-naïve patients and promoted notable durable disease control and prolonged overall survival in this pre-treated population.

The combination of 2-acetylnaphtho[2,3-b]furan-4,9-dione and paclitaxel was well tolerated. As shown in Table 6, the grade 3 gastrointestinal adverse events included diarrhea (N=2, 4.9%), abdominal pain (N=2, 4.9%), and nausea (N=1, 2.4%). These events were rapidly reversible.

TABLE 6
Adverse Events Related to Protocol Therapy (N = 41)

| | | Grade 1 | | Grade 2 | | Grade 3 | |
|---|---|---|---|---|---|---|---|
| System | Event Term | # | % | # | % | # | % |
| Gastrointestinal | Diarrhea | 27 | 65.9% | 12 | 29.3% | 2 | 4.9% |
| | Nausea | 18 | 43.9% | 4 | 9.8% | 1 | 2.4% |
| | Abdominal Pain | 11 | 26.8% | 6 | 14.6% | 2 | 4.9% |
| | Vomiting | 7 | 17.1% | 3 | 7.3% | 0 | 0.0% |
| | Flatulence | 1 | 2.4% | 0 | 0.0% | 0 | 0.0% |
| | Mucositis Oral | 0 | 0.0% | 0 | 0.0% | 1 | 2.4% |
| Constitutional | Fatigue | 11 | 26.8% | 7 | 17.1% | 0 | 0.0% |
| | Edema Limbs | 4 | 9.8% | 0 | 0.0% | 0 | 0.0% |
| | Lymphocyte Count Decreased | 0 | 0.0% | 1 | 2.4% | 1 | 2.4% |
| | Creatinine Increased | 1 | 2.4% | 0 | 0.0% | 0 | 0.0% |
| | AST Increased | 1 | 2.4% | 0 | 0.0% | 0 | 0.0% |
| | ALT Increased | 1 | 2.4% | 0 | 0.0% | 0 | 0.0% |
| | Fever | 0 | 0.0% | 1 | 2.4% | 0 | 0.0% |
| | Flu Like Symptoms | 0 | 0.0% | 1 | 2.4% | 0 | 0.0% |
| Metabolism - Nutrition | Anorexia | 8 | 19.5% | 3 | 7.3% | 0 | 0.0% |
| | Dehydration | 1 | 2.4% | 2 | 4.9% | 0 | 0.0% |
| | Hypomagnesemia | 2 | 4.9% | 0 | 0.0% | 0 | 0.0% |
| | Hypokalemia | 1 | 2.4% | 0 | 0.0% | 0 | 0.0% |
| | Hypoalbuminemia | 1 | 2.4% | 0 | 0.0% | 0 | 0.0% |
| | Hyponutremia | 1 | 2.4% | 0 | 0.0% | 0 | 0.0% |
| Renal and Urinary | Urine Discoloration | 4 | 9.8% | 0 | 0.0% | 0 | 0.0% |
| | Proteinuria | 2 | 4.9% | 0 | 0.0% | 0 | 0.0% |
| Hematologic | Anemia | 1 | 2.4% | 2 | 4.9% | 1 | 2.4% |
| | Leukopenia | 1 | 2.4% | 0 | 0.0% | 0 | 0.0% |
| Neuro-Psychiatric | Dizziness | 1 | 2.4% | 1 | 2.4% | 0 | 0.0% |
| | Peripheral Motor Neuropathy | 1 | 2.4% | 0 | 0.0% | 0 | 0.0% |
| Skin | Rash Maculo-Papular | 2 | 4.9% | 0 | 0.0% | 0 | 0.0% |
| Injury | Fall | 1 | 2.4% | 0 | 0.0% | 0 | 0.0% |
| Musculoskeletal | Arthralgia | 1 | 2.4% | 0 | 0.0% | 0 | 0.0% |
| Vascular | Hypotension | 1 | 2.4% | 0 | 0.0% | 0 | 0.0% |
| Other | Mucositis, NDS | 0 | 0.0% | 1 | 2.4% | 0 | 0.0% |

In sum, the disclosed combination therapy provided for effective anticancer activity and demonstrated the recommended phase 2 dose (PR2D) of 2-acetylnaphtho[2,3-b]furan-4,9-dione was 480 mg BID.

### Example 4

The effects of 2-acetylnaphtho[2,3-b]furan-4,9-dione in combination with paclitaxel in patients with metastatic triple negative breast cancer (TNBC) who progressed on prior systemic therapy (including prior taxanes) were studied in a phase Ib/II study to assess the combination's safety, tolerability, and preliminary anti-cancer activity.

Patients received oral administration of 2-acetylnaphtho[2,3-b]furan-4,9-dione twice daily together with paclitaxel. For example, 2-acetylnaphtho[2,3-b]furan-4,9-dione was administered at a dose of 480 mg BID in combination with paclitaxel at 80 mg/m² administered weekly as an IV infusion for 3 out every 4 weeks.

A sample size of 40 set the bounds of the 90% Cl at ±10% to 14%, assuming a disease control rate (DCR) of 60% to 80%. In this example, DCR was the proportion of patients with stable disease (SD) for at least 8 weeks, or objective partial (PR) or complete response (CR) per RECIST 1.1.

The 35 enrolled patients received a median of 4 prior lines of therapy, including 33 patients (94%) who had progressed on prior taxane-based regimens.

The combination of 2-acetylnaphtho[2,3-b]furan-4,9-dione together with paclitaxel demonstrated anti-cancer activity in patients with TNBC. For the evaluable patients (N=32), for example, the disease control rate (DCR) was 63% and overall response rate (ORR) was 19%.

For the intent to treat population (N=35), the median progression-free survival (mPFS) was 10.6 weeks and median overall survival (mOS) was 37 weeks.

The combination of 2-acetylnaphtho[2,3-b]furan-4,9-dione plus weekly paclitaxel was well tolerated without dose-limiting toxicity. This therapy also exhibited a safety profile similar to that of each regimen as monotherapy. Grade 3 adverse events were rapidly reversible and included diarrhea (N=3), as well as nausea, vomiting, anorexia, abdominal pain, and fatigue (N=1 each).

This data shows 2-acetylnaphtho[2,3-b]furan-4,9-dione plus weekly paclitaxel was safe, tolerable, and surprisingly produced promising signs of anti-cancer activity in patients with heavily pretreated TNBC who had progressed following treatment with taxane-based regimens. Without being limited to any particular theory, the presence of 2-acetylnaphtho[2,3-b]furan-4,9-dione appeared to re-sensitize the patients to the paclitaxel treatment even when these patients had developed or started to develop resistance to taxane-based regimens.

In addition, patients were examined to determine whether cancer stem cell biomarkers were predictive of treatment outcome. Patients who were positive for the cancer stem cell marker pStat3 consistently exhibited longer median progression-free survival (PFS) and overall survival (OS) when treated with 2-acetylnaphtho[2,3-b]furan-4,9-dione in combination with paclitaxel compared to patients who were negative for pStat3. Without being limited to any particular theory, it would appear that pStat3 served as a predictive biomarker for prolonged survival.

### Example 5

The effects of 2-acetylnaphtho[2,3-b]furan-4,9-dione in combination with paclitaxel in patients with epithelial ovarian, fallopian tube, or peritoneal cancer were studied in a phase Ib/II study to assess the combination's safety, tolerability, and preliminary anti-cancer activity. A recommended phase 2 dose (RP2D) expansion study of 2-acetylnaphtho[2,3-b]furan-4,9-dione in combination with paclitaxel included patients with platinum resistant ovarian cancer (PROC). This study enrolled patients with advanced epithelial ovarian, fallopian tube, or peritoneal cancer who progressed on a prior taxane-based regimen, and who were resistant or refractory to platinum therapy.

Patients received oral administration of 2-acetylnaphtho[2,3-b]furan-4,9-dione twice daily together with paclitaxel. Specifically, 2-acetylnaphtho[2,3-b]furan-4,9-dione was administered at a dose of 240 mg to 480 mg BID in combination with paclitaxel at 80 mg/m² administered weekly as an IV infusion 3 out every 4 weeks.

A sample size of 40 set the bounds of the 90% Cl at ±10% to 14%, assuming a disease control rate (DCR) of 60% to 80%. In this example, DCR was the proportion of patients with stable disease (SD) for at least 8 weeks, or objective partial (PR) or complete response (CR) per RECIST 1.1.

In total, 56 patients were enrolled after a median 4 prior lines of therapy, including prior taxanes (92% paclitaxel only, 4% docetaxel only, 4% paclitaxel and docetaxel).

Anti-cancer activity was observed, as the evaluable patients (N=40) had a 68% DCR. Moreover, 40% of the patients experienced tumor regression and the overall response rate (ORR) (PR+CR) was 25%, including 1 patient with CR. Prior to being evaluated, 2 patients withdrew due to neuropathy, 6 for other adverse events, 5 for deterioration, 2 for non-compliance, and 1 for myocardial infarction (unrelated).

In the intent to treat (ITT) patients (N=56), DCR was 48% and overall response rate was 18%. Additionally, the median progression-free survival (mPFS) was 15 weeks and median overall survival (mOS) was 38 weeks.

In patients with up to 2 prior lines of therapy (N'11), the overall response rate was 45%.

The combination of 2-acetylnaphtho[2,3-b]furan-4,9-dione and paclitaxel was well tolerated without dose-limiting toxicity and the safety profile was similar to that of each regimen as monotherapy. Grade 3 adverse events included rapidly-reversible diarrhea (18%), vomiting (7%), abdominal pain (7%), nausea (5%), dehydration (<4%), and fatigue (<4%). Eighty percent (80%) of patients with grade 3 adverse events continued the study at a reduced dose.

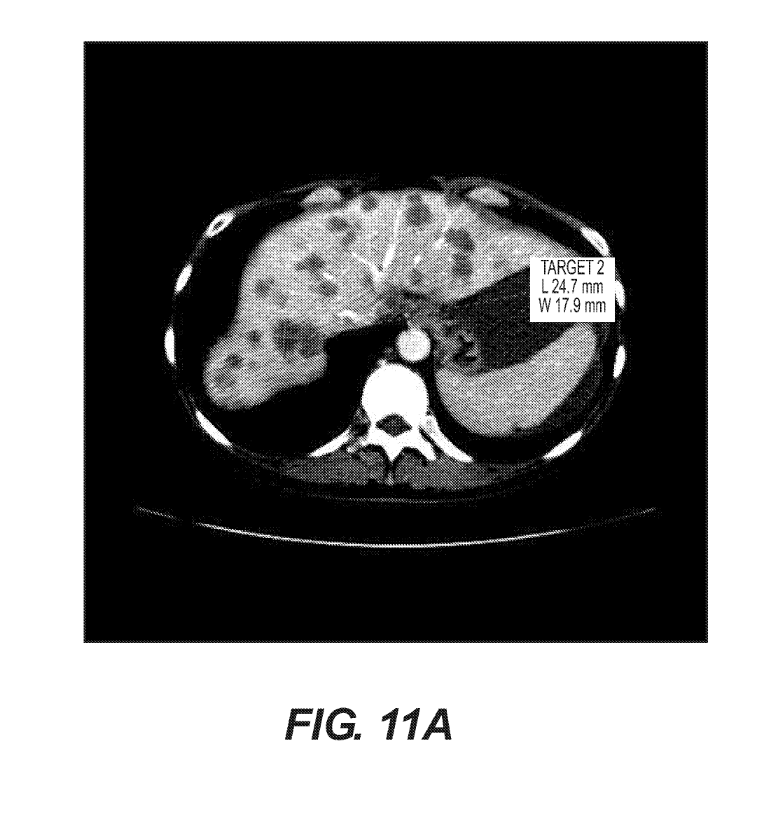

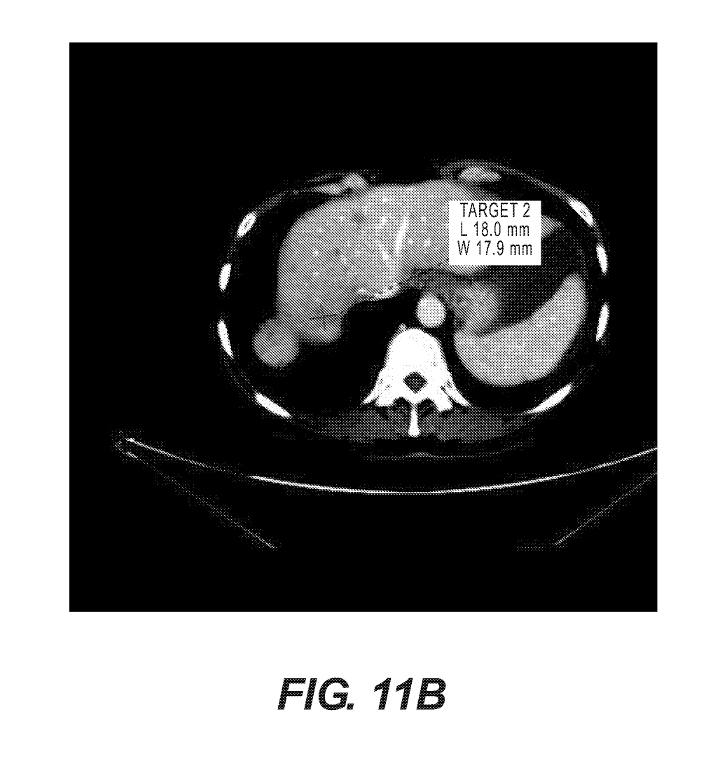

In addition, among the patients in the clinical trial, one with marked liver metastasis, profound ascites, and a CA-125 of 2000 (FIG. 11A) showed 28% regression at the 8^{th} week and 49% regression at the 16^{th} week (FIG. 11B) and a CA-125 of 102 at the 16^{th} week.

Accordingly, 2-acetylnaphtho[2,3-b]furan-4,9-dione at a dose of 240 to 480 mg BID was safely combined with weekly paclitaxel to promote anti-cancer activity. Specifically, the disclosed combination demonstrated acceptable tolerability in patients with heavily pretreated ovarian cancer, and surprisingly included patients with heavily pretreated PROC that had progressed on prior taxane-based regimens. Furthermore, complete and partial response, durable disease control, prolonged progression free survival, and overall survival was observed. Without being limited to any particular theory, the presence of 2-acetylnaphtho[2,3-b]furan-4,9-dione appeared to re-sensitize the patients to the paclitaxel treatment even when these patients had developed or started to develop resistance to taxane-based regimens.

### Example 6

The effects of 2-acetylnaphtho[2,3-b]furan-4,9-dione in combination with paclitaxel were clinically evaluated. In a phase Ib dose escalation study in patients with advanced solid tumors, 2-acetylnaphtho[2,3-b]furan-4,9-dione plus weekly paclitaxel was well tolerated. Phase II accrual to disease-specific cohorts included patients with advanced, heavily pre-treated metastatic non-small cell lung cancer (NSCLC).

Patients with metastatic squamous or non-squamous NSCLC who had progressed on prior systemic therapy were enrolled to assess the safety, tolerability, and preliminary anti-cancer activity of 2-acetylnaphtho[2,3-b]furan-4,9-dione plus weekly paclitaxel.

Patients received oral administration of 2-acetylnaphtho[2,3-b]furan-4,9-dione twice daily together with paclitaxel. For example, 2-acetylnaphtho[2,3-b]furan-4,9-dione was administered at a starting dose of 240 mg BID in combination with paclitaxel at 80 mg/m² administered weekly as an IV infusion 3 out every 4 weeks.

A sample size of 40 set the bounds of the 90% Cl at ±10% to 14%, assuming a disease control rate (DCR) of 60% to 80%. In this example, DCR was the proportion with patients with stable disease (SD) for at least 8 weeks, or objective partial (PR) or complete response (CR) per RECIST 1.1.

In this study, 27 patients enrolled with a median number of 3 prior lines of systemic treatment. Twenty-six (26) of the enrolled patients (96%) had received prior taxane-based therapy. All of those patients had progressed on the prior taxane therapy.

The combination treatment disclosed herein exhibited anti-cancer activity. For the evaluable patients (N=19), the DCR was 79%. Additionally, 37% of the patients experienced tumor regression and the objective partial response (PR) was 16%.

For evaluable non-squamous patients (N=15), the DCR was 87%. Tumor regression occurred in 47% of the patients and PR in 20%.

Overall, DCR was 56% in the intent to treat (ITT) patients (N=27). Tumor regression occurred in 26% of the patients and PR in 11%. The median progression free survival (mPFS) was 16 weeks and median overall survival (mOS) was 34 weeks.

For non-squamous patients (intention-to-treat (ITT), N=22), mPFS was 17 weeks and mOS was 37 weeks.

The combination treatment disclosed herein was well tolerated. Related grade 3 adverse events, including diarrhea (N=1) and hyponatremia (N=1), were rapidly reversible.

In summary, 2-acetylnaphtho[2,3-b]furan-4,9-dione at a starting dose of 240 mg BID in combination with paclitaxel resulted in anti-cancer activity. Specifically, objective response, tumor regression, durable disease control, prolonged progression free survival, and overall survival were observed in patients with heavily pretreated NSCLC. Accordingly, these results demonstrated the safety, tolerability, and anti-cancer activity of 2-acetylnaphtho[2,3-b]furan-4,9-dione in combination with paclitaxel in taxane refractory patients. Without being limited to any particular theory, the presence of 2-acetylnaphtho[2,3-b]furan-4,9-dione appeared to re-sensitize the patients to the paclitaxel treatment even when these patients had developed or started to develop resistance to taxane-based regimens.

In addition, patients were examined to determine whether cancer stem cell biomarkers were predictive of treatment outcome. Patients who were positive for the cancer stem cell marker pStat3 consistently exhibited longer survival (OS) when treated with 2-acetylnaphtho[2,3-b]furan-4,9-dione in combination with paclitaxel compared to patients who were negative for pStat3. Without being limited to any particular theory, it would appear that pStat3 served as a predictive biomarker for prolonged survival.

### Example 7

The effects of 2-acetylnaphtho[2,3-b]furan-4,9-dione in combination with paclitaxel in patients with melanoma, small cell lung cancer, or cholangiocarcinoma were studied in a phase Ib/II study to assess the combination's safety, tolerability, and preliminary anti-cancer activity. A recommended phase 2 dose (RP2D) expansion study of 2-acetylnaphtho[2,3-b]furan-4,9-dione in combination with paclitaxel included patients with these cancers. This study enrolled patients with advanced melanoma, small cell lung cancer, or cholangiocarcinoma.

Patients received oral administration of 2-acetylnaphtho[2,3-b]furan-4,9-dione twice daily together with paclitaxel. Specifically, 2-acetylnaphtho[2,3-b]furan-4,9-dione was administered at a dose of 240 mg to 480 mg BID in combination with paclitaxel at 80 mg/m² administered weekly as an IV infusion 3 out every 4 weeks.

A sample size of 40 set the bounds of the 90% Cl at ±10% to 14%, assuming a disease control rate (DCR) of 60% to 80%. In this example, DCR was the proportion of patients with stable disease (SD) for at least 8 weeks, or objective partial (PR) or complete response (CR) per RECIST 1.1.

The patients with melanoma, small cell lung cancer, and cholangiocarcinoma showed evaluable DCRs of 73%, 38%, and 41%, respectively; and the patients with melanoma and small cell lung cancer showed evaluable ORR of 9% and 14%, respectively. Among the patients with small cell lung cancer, 29% of the evaluable patients showed regression.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described accordingly, all suitable modifications and equivalents may be resorted to, falling within the scope of the present disclosure.

What is claimed is:

1. A method for treating lung cancer, breast cancer, ovarian cancer, pancreatic cancer, melanoma, small cell lung cancer, or cholangiocarcinoma in a subject comprising administering to a subject
a therapeutically effective amount of at least one compound of formula (I): 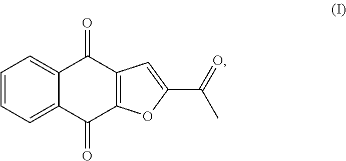 [SMILES: CC(=O)c1cc2c(o1)C(=O)c1ccccc1C2=O]
and
a therapeutically effective amount of at least one paclitaxel compound chosen from paclitaxel, pharmaceutically acceptable salts thereof, and solvates of any of the foregoing,
wherein the at least one paclitaxel compound is administered weekly.

2. The method according to claim 1, wherein the cancer progressed on at least one prior taxane chemotherapy regimen.

3. A method for resensitizing a subject to a paclitaxel chemotherapy regimen comprising administering to a subject whose lung cancer, breast cancer, ovarian cancer, pancreatic cancer, melanoma, small cell lung cancer, or cholangiocarcinoma progressed on prior taxane chemotherapy
a therapeutically effective amount of at least one of formula (I):

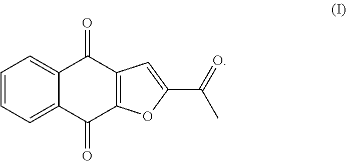 [SMILES: CC(=O)c1cc2c(o1)C(=O)c1ccccc1C2=O]

4. A method of simultaneously:
(i) inhibiting, reducing, and/or diminishing survival and/or self-renewal of cancer stem cells, and
(ii) inhibiting, reducing, and/or diminishing survival and/or proliferation heterogeneous cancer cells chosen from lung cancer, breast cancer, ovarian cancer, pancreatic cancer, melanoma, small cell lung cancer, and cholangiocarcinoma cells in a subject, comprising administering to a subject in need thereof:
(i) a therapeutically effective amount of at least one compound of formula (I) 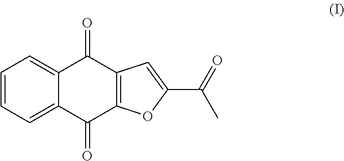 [SMILES: CC(=O)c1cc2c(o1)C(=O)c1ccccc1C2=O]
and
(ii) a therapeutically effective amount of at least one paclitaxel compound chosen from paclitaxel, pharmaceutically acceptable salts thereof, and solvates of any of the foregoing.

5. A method preventing cancer relapses in a subject comprising administering to a subject
a therapeutically effective amount of at least one compound of formula (I): 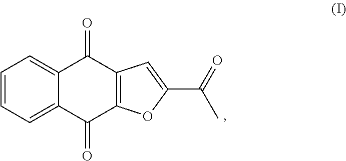 [SMILES: CC(=O)c1cc2c(o1)C(=O)c1ccccc1C2=O]
and
a therapeutically effective amount of at least one paclitaxel compound chosen from paclitaxel, pharmaceutically acceptable salts thereof, and solvates of any of the foregoing,
wherein the at least one paclitaxel compound is administered weekly.

6. The method according any one of claims 1-5, wherein the at least one compound of formula (I) is chosen from compounds having formula (I) 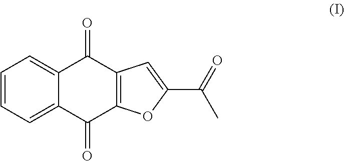 [SMILES: CC(=O)c1cc2c(o1)C(=O)c1ccccc1C2=O]
prodrugs, derivatives, pharmaceutically acceptable salts of any of the foregoing, and solvates of any of the foregoing.

7. The method according to any one of claims 1-6, wherein the cancer is pancreatic cancer.

8. The method according to claim 7, wherein the pancreatic cancer is metastatic pancreatic adenocarcinoma.

9. The method according to any one of claims 1-6, wherein the cancer is breast cancer.

10. The method according to claim 9, wherein the breast cancer is advanced triple negative breast cancer.

11. The method according to any one of claims 1-6, wherein the cancer is ovarian cancer.

12. The method according to claim 11, wherein the ovarian cancer is platinum-resistant ovarian cancer.

13. The method according to any one of claims 1-6, wherein the cancer is lung cancer.

14. The method according to claim 13, wherein the cancer is non-small cell lung cancer.

15. The method according to any one of claims 1-6, wherein the at least one compound of formula (I) is administered at a dose of about 480 mg per day.

16. The method according to claim 15, wherein the at least one compound of formula (I) is administered in a divided dose.

17. The method according to any one of claims 1-6, wherein the at least one compound of formula (I) is administered at a dose of about 240 mg, about 480 mg, or about 500 mg twice daily.

18. The method according to any one of claims 1-6, wherein the at least one paclitaxel compound is administered as an about 80 mg/m² infusion.

19. The method according to any one of claims 1-6, wherein the cancer is advanced, metastatic, unresectable, or recurrent.

20. A kit comprising
at least one paclitaxel compound chosen from paclitaxel, pharmaceutically acceptable salts thereof, and solvates of any of the foregoing, and
at least one compound of formula (I): 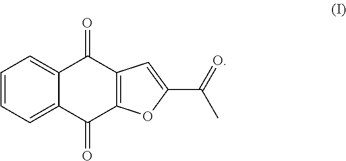 [SMILES: CC(=O)c1cc2c(o1)C(=O)c1ccccc1C2=O]
